SEQUENCE LISTING (53 sequences)

SEQ ID NO: 1 - DNA, 375 bp, synthetic construct
  misc_feature 1..375: note = 3B9 VL1-VH nucleic acid sequence
  source 1..375: mol_type = other DNA
caggtgcagctggtgcagtctggggctgaggtgaagaagcctgggtcctcggtgaaggtctcctgcaaggcttctggaggcaccttcagcagctatgctatcagctgggtgcgccaggcccctggacaagggcttgagtggatgggagggatcatccctctctttgatacaacaaagtacgcacagcagttccagggcagagtcacgattaccgcggacgaatccacgagcacagcctacatggagctgagcagcctgagatctgaggacacggccgtattttactgtgcgagagatcgggatattttgactgattattatcccatgggcggtatggacgtctggggccaagggaccacggtcaccgtctcctca

SEQ ID NO: 2 - protein, 125 aa, synthetic construct
  REGION 1..125: note = 3B9 VL1-VH amino acid sequence
QVQLVQSGAEVKKPGSSVKVSCKASGGTFSSYAISWVRQAPGQGLEWMGGIIPLFDTTKYAQQFQGRVTITADESTSTAYMELSSLRSEDTAVFYCARDRDILTDYYPMGGMDVWGQGTTVTVSS

SEQ ID NO: 3 - DNA, 324 bp, synthetic construct
  misc_feature 1..324: note = 39B9 VL1-VL nucleic acid sequence
  source 1..324: mol_type = other DNA
gccatccagttgacccagtctccatcctccctgtctgcatctgtaggagacagagtcaccatcacttgccgggcaagtcagggcattagcagtgttttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtctaatagttacccgctcactttcggcggagggaccaaggtggagatcaaacgt

SEQ ID NO: 4 - protein, 108 aa, synthetic construct
  REGION 1..108: note = 39B9 VL1-VL amino acid sequence
AIQLTQSPSSLSASVGDRVTITCRASQGISSVLAWYQQKPGKAPKLLIYDASSLESGVPSRFSGSGSGTDFTLTISSLQPEDFATYYCQQSNSYPLTFGGGTKVEIKR

SEQ ID NO: 5 - DNA, 324 bp, synthetic construct
  misc_feature 1..324: note = 39B9 VL5-VL nucleic acid sequence
  source 1..324: mol_type = other DNA
gacatcctgatgacccagtctccatcctccctgtctgcatctgtaggagacagagtcaccatcacttgtcgggcgagtcaggatattagcagctggttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtctaatagttacccgctcactttcggcggagggaccaaggtggagatcaaacga

SEQ ID NO: 6 - protein, 108 aa, synthetic construct
  REGION 1..108: note = 39B9 VL5-VL amino acid sequence
DILMTQSPSSLSASVGDRVTITCRASQDISSWLAWYQQKPGKAPKLLIYDASSLESGVPSRFSGSGSGTDFTLTISSLQPEDFATYYCQQSNSYPLTFGGGTKVEIKR

SEQ ID NO: 7 - DNA, 357 bp, synthetic construct
  misc_feature 1..357: note = 12A VH nucleic acid sequence
  source 1..357: mol_type = other DNA
caggtgcagctggtggagtcttggggaggcgtggtccagcctgggaggtccctgagactctcctgtgcagcgtctggattcaccttcagtaactatgacatgtactgggtccgccaggctccaggcaaggggctggagtgggtggcagttatattagatgatggaaataataattactacgcagactccgtgaagggccgattcaccatctccagagacaattccaagaaaaaggtgtatctgcaaatgaatagcctgagagctgaggacacggctgtgtattactgtgtgagagcgtcccctaactggggtcttcttgacttctggggccagggaaccctggtcaccgtctcgagt

SEQ ID NO: 8 - protein, 119 aa, synthetic construct
  REGION 1..119: note = 12A VH amino acid sequence
QVQLVESWGGVVQPGRSLRLSCAASGFTFSNYDMYWVRQAPGKGLEWVAVILDDGNNNYYADSVKGRFTISRDNSKKKVYLQMNSLRAEDTAVYYCVRASPNWGLLDFWGQGTLVTVSS

SEQ ID NO: 9 - DNA, 324 bp, synthetic construct
  misc_feature 1..324: note = 12A VL nucleic acid sequence
  source 1..324: mol_type = other DNA
gaaattgtgttgacacagtctccatcctcactgtctgcatctgtaggagacagagtcaccatcacttgtcgggcgagtcagggtattagcagctggttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgt

SEQ ID NO: 10 - protein, 108 aa, synthetic construct
  REGION 1..108: note = 12A VL amino acid sequence
EIVLTQSPSSLSASVGDRVTITCRASQGISSWLAWYQQKPGKAPKLLIYDASSLESGVPSRFSGSGSGTDFTLTISSLQPEDFATYYCQQFNSYPITFGQGTRLEIKR

SEQ ID NO: 11 - DNA, 357 bp, synthetic construct
  misc_feature 1..357: note = 5C VH nucleic acid sequence
  source 1..357: mol_type = other DNA
caggtgcagctggtgcagtctgggggaggcgtggtccagcctgggaggtccctgagactctcctgtgcagcctctggattcatcttcagtagctatgacatgtactgggtccgccaggctccaggcaaggggctggagtgggtggcagttatattatatgatggaaataataaatactacgcagactccgtgaagggccgattcaccatctccagagacaattccaagaacacggtgtatctgcaaatgaacagcctgagagctgaggacacggctgtgtattactgtgtgagagcgtcccctaactggggtctttttgacttctggggccagggaaccctggtcaccgtctcgagt

SEQ ID NO: 12 - protein, 119 aa, synthetic construct
  REGION 1..119: note = 5C VH amino acid sequence
QVQLVQSGGGVVQPGRSLRLSCAASGFIFSSYDMYWVRQAPGKGLEWVAVILYDGNNKYYADSVKGRFTISRDNSKNTVYLQMNSLRAEDTAVYYCVRASPNWGLFDFWGQGTLVTVSS

SEQ ID NO: 13 - DNA, 324 bp, synthetic construct
  misc_feature 1..324: note = 5C VL nucleic acid sequence
  source 1..324: mol_type = other DNA
gacatccagatgacccagtctccatcctccctgtctgcatctgtaggagacagagtcaccatcacttgccgggcaagtcagggcattagcagtgatttagcctggtatcagcagaaaccagggaaagctcctaagctcctgatgtatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgt

SEQ ID NO: 14 - protein, 108 aa, synthetic construct
  REGION 1..108: note = 5C VL amino acid sequence
DIQMTQSPSSLSASVGDRVTITCRASQGISSDLAWYQQKPGKAPKLLMYDASSLESGVPSRFSGSGSGTDFTLTISSLQPEDFATYYCQQFNSYPITFGQGTRLEIKR

SEQ ID NO: 15 - protein, 5 aa, synthetic construct
  REGION 1..5: note = 39B9 VH CDR1
SYAIS

SEQ ID NO: 16 - protein, 17 aa, synthetic construct
  REGION 1..17: note = 39B9 VH CDR2
GIIPLFDTTKYAQQFQG

SEQ ID NO: 17 - protein, 19 aa, synthetic construct
  REGION 1..19: note = 39B9 VH CDR3
CARDRDILTDYYPMGGMDV

SEQ ID NO: 18 - protein, 5 aa, synthetic construct
  REGION 1..5: note = 12A VH CDR1
NYDMY

SEQ ID NO: 19 - protein, 17 aa, synthetic construct
  REGION 1..17: note = 12A VH CDR2
VILDDGNNNYYADSVKG

SEQ ID NO: 20 - protein, 13 aa, synthetic construct
  REGION 1..13: note = 12A VH CDR3
CVRASPNWGLLDF

SEQ ID NO: 21 - protein, 5 aa, synthetic construct
  REGION 1..5: note = 5C VH CDR1
SYDMY

SEQ ID NO: 22 - protein, 17 aa, synthetic construct
  REGION 1..17: note = 5C VH CDR2
VILYDGNNKYYADSVKG

SEQ ID NO: 23 - protein, 13 aa, synthetic construct
  REGION 1..13: note = 5C VH CDR3
CVRASPNWGLFDF

SEQ ID NO: 24 - protein, 11 aa, synthetic construct
  REGION 1..11: note = 39B9 VL1 VL CDR1
RASQGISSVLA

SEQ ID NO: 25 - protein, 7 aa, synthetic construct
  REGION 1..7: note = IL-6 antibody VL CDR2
DASSLES

SEQ ID NO: 26 - protein, 9 aa, synthetic construct
  REGION 1..9: note = 39B9 VL1 VL CDR3
QQSNSYPLT

SEQ ID NO: 27 - protein, 11 aa, synthetic construct
  REGION 1..11: note = 39B9 VL5 VL CDR1
RASQDISSWLA

SEQ ID NO: 28 - protein, 11 aa, synthetic construct
  REGION 1..11: note = 12A VL CDR1
RASQGISSWLA

SEQ ID NO: 29 - protein, 9 aa, synthetic construct
  REGION 1..9: note = 12A VL CDR3
QQSNSYPIT

SEQ ID NO: 30 - protein, 11 aa, synthetic construct
  REGION 1..11: note = 5C VL CDR1
RASQGISSVDA

SEQ ID NO: 31 - protein, 9 aa, synthetic construct
  REGION 1..9: note = VL CDR3 motif
  VARIANT 4: note = X is asparagine or glutamine
QQSXSYPLT

SEQ ID NO: 32 - protein, 17 aa, synthetic construct
  REGION 1..17: note = VH CDR2 motif
  VARIANT 5: note = X is leucine or alanine
  VARIANT 7: note = X is aspartic acid or glutamic acid
  VARIANT 14: note = X is glutamine or lysine
GIIPXFXTTKYAQXFQG

SEQ ID NO: 33 - protein, 16 aa, synthetic construct
  REGION 1..16: note = VH CDR3 motif
  VARIANT 11: note = X is methionine or leucine
DRDILTDYYPXGGMDV

SEQ ID NO: 34 - protein, 8 aa, synthetic construct
  REGION 1..8: note = FRW3 mofitf
  VARIANT 4: note = X is phenylalanine or tyrosine
TAVXYCAR

SEQ ID NO: 35 - protein, 16 aa, synthetic construct
  REGION 1..16: note = NI-1201-WT VH CDR3
DRDILTDYYPMGGMDV

SEQ ID NO: 36 - protein, 8 aa, synthetic construct
  REGION 1..8: note = NI-1201-WT FRW3
TAVFYCAR

SEQ ID NO: 37 - protein, 17 aa, synthetic construct
  REGION 1..17: note = NI-1201-A VL CDR3
GIIPLFDTTKYAQKFQG

SEQ ID NO: 38 - protein, 8 aa, synthetic construct [sequence omitted]

SEQ ID NO: 39 - protein, 16 aa, synthetic construct
  REGION 1..16: note = NI-1201-B VH CDR3
DRDILTDYYPLGGMDV

SEQ ID NO: 40 - protein, 17 aa, synthetic construct
  REGION 1..17: note = NI-1201-C VH CDR2
GIIPAFETTKYAQKFQG

SEQ ID NO: 41 - protein, 9 aa, synthetic construct
  REGION 1..9: note = NI-1201-D VL CDR3
QQSQSYPLT

SEQ ID NO: 42 - protein, 5 aa, synthetic construct
  REGION 1..5: note = anti-CD3 CDRH1
GYGMH

SEQ ID NO: 43 - protein, 17 aa, synthetic construct
  REGION 1..17: note = anti-CD3 CDRH2
VIWYDGSKKYYVDSVKG

SEQ ID NO: 44 - protein, 9 aa, synthetic construct
  REGION 1..9: note = anti-CD3 CDRH3
QMGYWHFDL

SEQ ID NO: 45 - protein, 11 aa, synthetic construct
  REGION 1..11: note = anti-CD3 CDRL1
RASQSVSSYLA

SEQ ID NO: 46 - protein, 7 aa, synthetic construct
  REGION 1..7: note = anti-CD3 CDRL2
DASNRAT

SEQ ID NO: 47 - protein, 10 aa, synthetic construct
  REGION 1..10: note = anti-CD3 CDRL3
QQRSNWPPLT

SEQ ID NO: 48 - protein, 118 aa, synthetic construct [sequence omitted]

SEQ ID NO: 49 - protein, 108 aa, synthetic construct [sequence omitted]

SEQ ID NO: 50 - protein, 448 aa, synthetic construct
  REGION 1..448: note = anti-CD3 HC
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 215 aa, synthetic construct
  REGION 1..215: note = anti-CD3 LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 454 aa, synthetic construct
  REGION 1..454: note = TZLS-501 heavy chain
QVQLVQSGAEVKKPGSSVKVSCKASGGTFSSYAISWVRQAPGQGLEWMGGIIPLFDTTKYAQKFQGRVTITADESTSTAYMELSSLRSEDTAVYYCARDRDILTDYYPMGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 214 aa, synthetic construct
  REGION 1..214: note = TZLS-501 light chain
AIQLTQSPSSLSASVGDRVTITCRASQGISSVLAWYQQKPGKAPKLLIYDASSLESGVPSRFSGSGSGTDFTLTISSLQPEDFATYYCQQSNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0125483 A1
(43) Pub. Date: May 7, 2026

(54) COMPOSITIONS OF IL-6/IL-6R ANTIBODIES AND METHODS OF USE THEREOF

(71) Applicant: Tiziana Life Sciences PLC, London (GB)

(72) Inventors: Kunwar SHAILUBHAI, Line Lexington, PA (US); Gabriele CERRONE, London (GB); Vaseem PALEJWALA, Scotch Plains, NJ (US); Jules S. JACOB, Perkasie, PA (US)

(21) Appl. No.: 19/209,623

(22) Filed: May 15, 2025

Related U.S. Application Data
(63) Continuation of application No. 18/906,776, filed on Oct. 4, 2024, now abandoned.
Application No. 18/906,776 is a continuation of application No. 17/197,603, filed on Mar. 10, 2021, now abandoned.
(60) Provisional application No. 62/987,837, filed on Mar. 10, 2020.
(60) Provisional application No. 63/006,612, filed on Apr. 7, 2020.
(60) Provisional application No. 63/014,800, filed on Apr. 24, 2020.

(51) Int. Cl. C07K 16/28; A61K 9/00; A61K 45/06; A61K 47/02; A61K 47/18; A61K 47/26; C07K 16/24

(52) U.S. Cl. CPC C07K 16/2866; A61K 9/0078; A61K 45/06; A61K 47/02; A61K 47/183; A61K 47/26; C07K 16/241; C07K 16/243; C07K 16/2809; C07K 16/2887; C07K 2317/565; C07K 2317/94

(57) ABSTRACT

The present disclosure provides compositions and methods for the treatment of coronavirus infections, such as SAR-CoV, SARS-CoV-2, and MERS-CoV. The compositions include antibodies targeting the IL-6 receptor complex, antibodies targeting CD3, dactinomycin, and combinations thereof. The methods of treatment include administration of antibodies and combination therapies to reduce or eliminate symptoms associated with coronavirus infection or pulmonary inflammatory disease.

### CROSS REFERENCE TO RELATED APPLICATIONS

This application is a continuation of U.S. application Ser. No. 18/906,776, filed Oct. 4, 2024, which is a continuation of U.S. application Ser. No. 17/197,603, filed Mar. 10, 2021, which claims the benefit of priority to U.S. Provisional Patent Application No. 62/987,837 filed on Mar. 10, 2020, U.S. Provisional Patent Application No. 63/006,612 filed on Apr. 7, 2020, and U.S. Provisional Patent Application No. 63/014,800 filed on Apr. 24, 2020, the contents of which are hereby incorporated by reference in their entireties.

### REFERENCE TO SEQUENCE LISTING

The Sequence Listing XML associated with this application is provided electronically in XML file format and is hereby incorporated by reference into the specification. The name of the XML file containing the Sequence Listing XML is “TIZI-022_C02US_SeqList_ST26-updated.xml”. The XML file is 73,922 bytes in size, created on Dec. 17, 2025 and is being submitted electronically via USPTO Patent Center.

### TECHNICAL FIELD

The disclosure relates generally to composition of monoclonal antibodies, e.g., fully human monoclonal antibodies, that recognize the IL-6/IL-6R complex, and methods of using same to treat coronaviruses, e.g., COVID-19, SARs, and MERS. Also included are compositions of dactinomycin and anti-CD3 antibodies for use in combination with the IL-6/IL-6R antibodies to treat coronaviruses, e.g., COVID-19, SARs, MERS and other pulmonary inflammatory diseases.

### BACKGROUND

Coronaviruses are enveloped non-segmented positive sense RNA viruses belonging to the family Coronaviridae. Coronaviruses can cause multiple system infections mainly respiratory tract infections in humans, such as severe acute respiratory syndrome (SARS) and Middle East respiratory syndrome (MERS). A novel coronavirus, herein referred to interchangeably as 2019-nCoV, SAR-CoV-2, or COVID-19, originating in Wuhan, China presents a potential respiratory viral pandemic to the world population. Current efforts are focused on containment and quarantine of infected individuals. This outbreak could be controlled with a protective vaccine to prevent COVID-19 infection but so far, no vaccine exists for treatment of this virus.

Interleukin 6 (IL-6) is a potent pleiotropic cytokine that regulates cell growth and differentiation and is also an important mediator of acute inflammatory responses. IL-6 exhibits its action via a receptor complex consisting of a specific IL-6 receptor (IL-6R) and a signal transducing subunit (gp130). Dysregulated IL-6 signaling has been implicated in the pathogenesis of many diseases, such as COVID-19 caused by coronavirus. Accordingly, there exists a need for therapies that neutralize the biological activities of IL-6 and/or IL-6R to treat and prevent coronavirus infections such as COVID-19.

### SUMMARY

In one aspect, the disclosure provides a method of treating, preventing, or alleviating a symptom of a coronavirus infection in a subject in need thereof comprising administering to the subject a composition comprising an IL-6R antibody.

In another aspect, the disclosure provides a method of treating, preventing, or alleviating a symptom of a coronavirus infection in a subject in need thereof comprising administering to the subject: a composition comprising an IL-6R antibody; and a composition comprising dactinomycin nanoparticles, wherein administration of the composition comprising IL-6R antibody and administration of the composition comprising dactinomycin nanoparticles can occur in any order or simultaneously.

In some embodiments, the coronavirus is COVID-19, SARS or MERS. In some embodiments, the symptom of a coronavirus infection is fever, cough, shortness of breath. In some embodiments, the subject has or is suspected of having a coronavirus infection. In some embodiments, the subject has been or thought to have been exposed to a coronavirus and has not yet developed symptoms of a coronavirus infection.

In some embodiments, the IL-6R antibody comprising a VH CDR1 region comprising the amino acid sequence of SEQ ID NO: 15, a VH CDR2 region comprising the amino acid sequence of SEQ ID NO: 37, a VH CDR3 region comprising the amino acid sequence of SEQ ID NO: 35, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26.

In some embodiments, the IL-6R antibody is tocilizumab or sarilumab.

In some embodiments, the IL-6R antibody is a monoclonal antibody. In some embodiments, the antibody is fully human.

The methods of the disclosure may further comprise administering an anti-TNFα antibody, an anti-CD20 antibody an anti-IFNγ antibody an anti-Granulocyte-Macrophage Colony-Stimulating Factor antibody or an anti-CD3 antibody.

In some embodiments, the composition comprising an IL-6R antibody is administered by inhalation, nasally, intravenously or any combination thereof. In some embodiments, the inhalation administration is by an inhaler or a nebulizer. In some embodiments, the inhaler or nebulizer comprises a composition comprising the IL-6R antibody, histidine, sodium chloride, and polysorbate 80. In some embodiments, the histidine is about 5 mM, about 10 mM, about 15 mM, about 20 mM, about 25 mM, about 30 mM, about 35 mM, or about 40 mM. In some embodiments, the histidine is about 25 mM. In some embodiments, the sodium chloride is about 50 mM, about 75 mM, about 100 mM, about 125 mM, about 150 mM, about 175 mM or about 200 mM. In some embodiments, the sodium chloride is about 125 mM. In some embodiments, the polysorbate 80 is about 0.01%, about 0.02%, about 0.03%, about 0.04%, about 0.05%, about 0.06%, about 0.07%, about 0.08%, about 0.09%, or about 0.1%. In some embodiments, the polysorbate 80 is about 0.02%. In some embodiments, the polysorbate 80 is about 0.05%. In some embodiments, the pharmaceutically acceptable composition has a pH of about 5.0, about 6.0, or about 7.0. In some embodiments, the pharmaceutically acceptable composition has a pH of about 6.0. In some embodiments, the IL-6R antibody is about 5 mg/mL, about 10 mg/mL, about 15 mg/mL, about 20 mg/mL, about 25 mg/mL, about 30 mg/mL, or about 35 mg/mL. In some embodiments, the IL-6R antibody is about 20 mg/mL. In some embodiments, the histidine is 25 mM, the sodium chloride is 125 mM, the polysorbate 80 is 0.02%, the pH is 6.0, and the IL-6R antibody is 20 mg/mL. In some embodiments, the histidine is 25 mM, the sodium chloride is 125 mM, the polysorbate 80 is 0.05%, the pH is 6.0, and the IL-6R antibody is 20 mg/mL.

In some embodiments, the methods of the disclosure comprise administering an anti-CD3 antibody. In some embodiments, the anti-CD3 antibody is foralumab.

In some embodiments, the methods of the disclosure further comprise administering to the subject an antiviral drug, an immune booster drug, vitamin C, Vitamin D or any combination thereof. In some embodiments, the antiviral drug is Remdesivir or Actinomycin D.

In some embodiments, the methods of the disclosure further comprise administering a broad spectrum and polymeric COVID-19 antigen.

In one aspect, the disclosure provides a method of producing anti-COVID-19 antibodies comprising administering to a subject a broad spectrum and polymeric COVID-19 antigen. In some embodiments, the subject does not have a coronavirus infection. In some embodiments, the methods further comprises isolating immunoglobulin from the subject, wherein the immunoglobin binds and neutralized COVID-19.

In one aspect, the disclosure provides a composition comprising the immunoglobulin isolated by the methods described herein. In a further aspect, the disclosure provides a methods of treating, preventing or alleviating a symptom of a coronavirus in a subject in need thereof comprising administering to the subject the composition comprising an immunoglobulin isolated by the methods described herein. In some embodiments, the composition is administered intravenously.

In one aspect, the disclosure provides a vaccine comprising a broad spectrum COVID-19 antigen described herein. In another aspect, the disclosure provides a method of preventing a coronavirus infection comprising administering to a subject the vaccine described herein.

In one aspect, the disclosure provides a composition comprising an IL-6R antibody, histidine, sodium chloride, and polysorbate 80. In some embodiments, the histidine is about 5 mM, about 10 mM, about 15 mM, about 20 mM, about 25 mM, about 30 mM, about 35 mM, or about 40 mM. In one embodiments, the histidine is about 25 mM. In some embodiments, the sodium chloride is about 50 mM, about 75 mM, about 100 mM, about 125 mM, about 150 mM, about 175 mM or about 200 mM. In one embodiment, the sodium chloride is about 125 mM. In some embodiments, the polysorbate 80 is about 0.01%, about 0.02%, about 0.03%, about 0.04%, about 0.05%, about 0.06%, about 0.07%, about 0.08%, about 0.09%, or about 0.1%. In one embodiments, the polysorbate 80 is about 0.02%. In one embodiment, the polysorbate 80 is about 0.05%. In some embodiments, the composition has a pH of about 5.0, about 6.0, or about 7.0. In some embodiments, composition has a pH of about 6.0. In some embodiments, the IL-6R antibody is about 5 mg/mL, about 10 mg/mL, about 15 mg/mL, about 20 mg/mL, about 25 mg/mL, about 30 mg/mL, or about 35 mg/mL. In some embodiments, the IL-6R antibody is about 20 mg/mL. In some embodiments, the histidine is 25 mM, the sodium chloride is 125 mM, the polysorbate 80 is 0.02%, the pH is 6.0, and the IL6R antibody is 20 mg/mL. In some embodiments, the histidine is 25 mM, the sodium chloride is 125 mM, the polysorbate 80 is 0.05%, the pH is 6.0, and the IL6R antibody is 20 mg/mL. In some embodiments, the IL-6R antibody comprises a VH CDR1 region comprising the amino acid sequence of SEQ ID NO: 15, a VH CDR2 region comprising the amino acid sequence of SEQ ID NO: 37, a VH CDR3 region comprising the amino acid sequence of SEQ ID NO: 35, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26.

In some embodiments, the composition is suitable for use in a nebulizer or inhaler.

In one aspect, the disclosure provides a composition described herein for use as a medicament in the treatment of a coronavirus infection or a pulmonary inflammatory disease in a subject in need thereof.

In one aspect, the disclosure provides a method of treating, preventing, or alleviating a symptom of a pulmonary inflammatory disease in a subject in need thereof comprising administering to the subject a composition comprising an IL-6R antibody.

In one aspect, the disclosure provides a method of treating, preventing, or alleviating a symptom of a pulmonary inflammatory disease in a subject in need thereof comprising administering to the subject: a composition comprising an IL-6R antibody; and a composition comprising dactinomycin nanoparticles, wherein administration of the composition comprising an IL-6R antibody and administration of the composition comprising dactinomycin nanoparticles can occur in any order or simultaneously.

In some embodiments, the pulmonary inflammatory disease is selected from acute respiratory distress syndrome (ARDS) or systemic pulmonary sclerosis.

### BRIEF DESCRIPTION OF THE DRAWINGS

FIG. 1 is an illustration of the therapeutic options during progression of COVID-19.

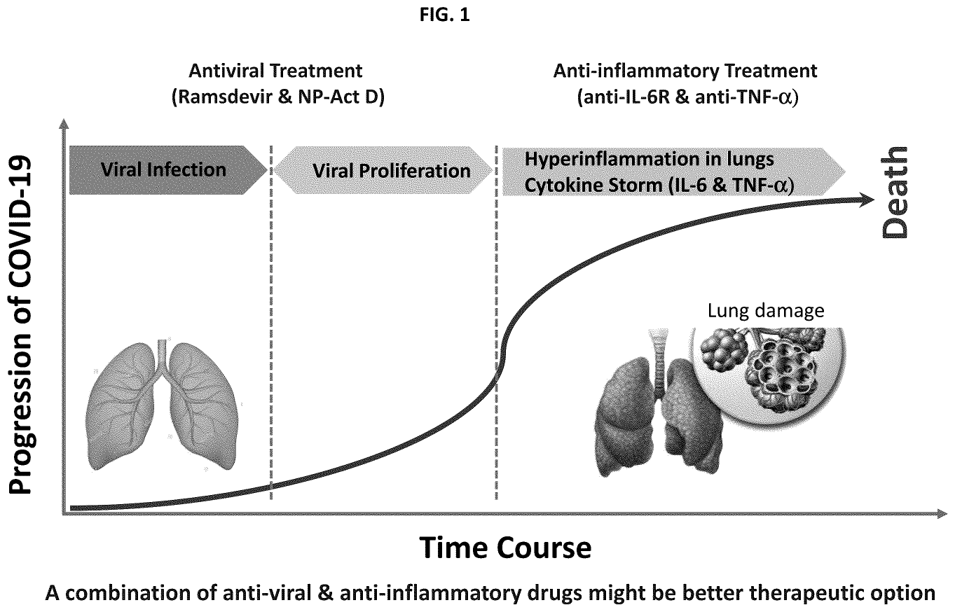

FIG. 2 is a diagram illustrating cis and trans IL-6 signaling, and the mechanism of inhibiting both types of IL-6 signaling mediated by the binding of an exemplary antibody.

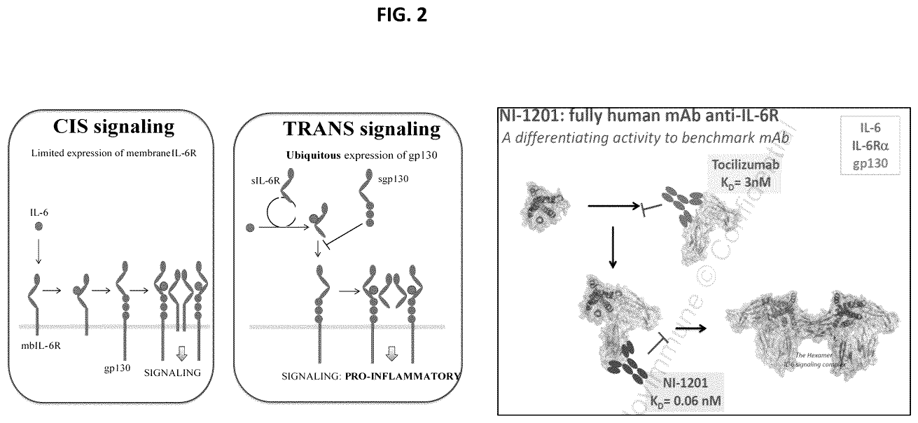

FIG. 3 is a chart comparing features of the IL-6 signaling targeting antibody, NI-1201, to approved IL-6 signaling targeted antibodies.

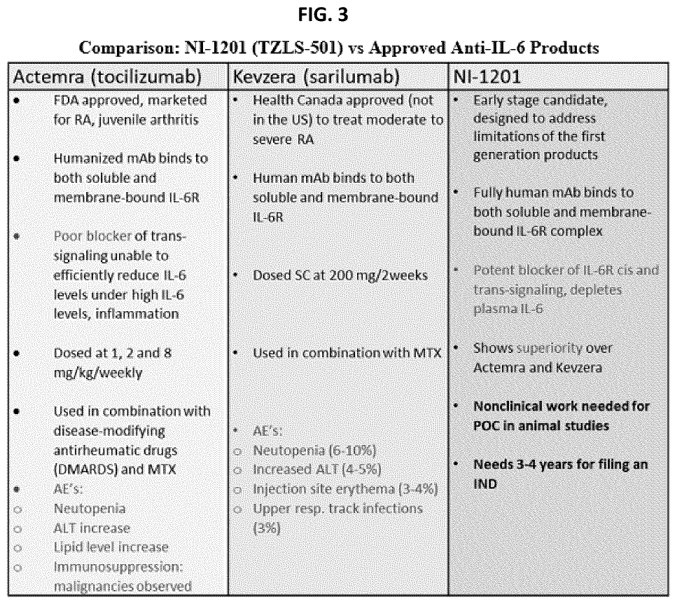

FIG. 4 is a drawing showing a handheld inhaler or nebulizer. A vial containing formulated drug solution may be inserted for aerosol delivery to patients.

FIG. 5 is a drawing showing the entry of COVID-19 and how it can be inhibited by sera from subjects immunized with S protein of COVID-19, which contains antibodies against S protein.

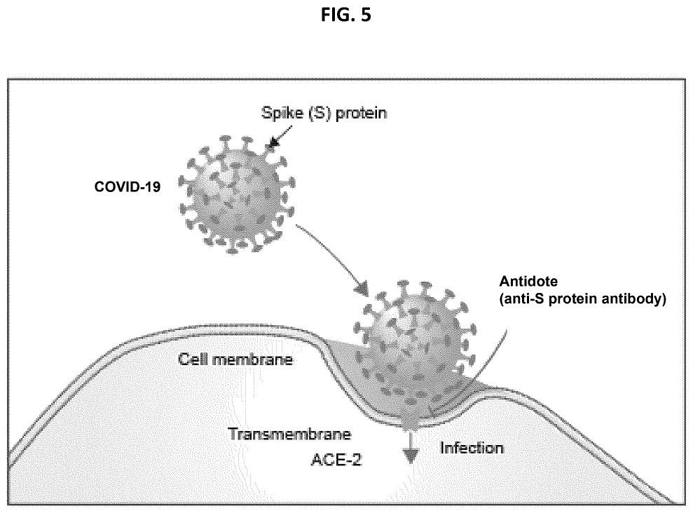

FIG. 6 is a chromatograph showing the relative abundance of aggregate particle size in an aerosolized formulation including an IL-6R targeting antibody, according to an embodiment. Chromatographs were generated by size exclusion chromatography showing HPLC.

FIG. 7 is a chromatograph showing the relative abundance of aggregate particle size in an aerosolized formulation including an IL-6R targeting antibody, according to an embodiment. Chromatographs were generated by size exclusion chromatography showing HPLC

FIG. 8 is a chromatograph showing the relative abundance of aggregate particle size in an aerosolized control formulation. Chromatographs were generating by size exclusion chromatography HPLC.

FIG. 9 is a series of histograms showing the particle size distribution of an example formulation containing an IL-6R antibody that is aerosolized in a nebulizer. The particle distribution was measured at the beginning, middle, and end of a nebulizer run.

### DETAILED DESCRIPTION

The present disclosure provides compositions of monoclonal antibodies that specifically bind the human IL-6/IL-6 receptor complex (“IL-6Rc”, “IL-6”, or “IL-6R”) for the treatment, prevention or alleviating a symptom of cytokine release syndrome (CRS).

CRS is triggered by drug therapy, an infectious disease or a non-infectious disease. Drug therapies that trigger CRS include for example, CAR-T cell therapy or monoclonal antibody therapy. Infectious diseases that trigger CRS include for example, a coronavirus disease, sepsis, Ebola, avian influenza, smallpox, pneumonia, or influenza. The coronavirus disease is coronavirus is COVID-19, SARS or MERS. Non-infectious inflammatory diseases that trigger CRS include for example, acute respiratory distress syndrome (ARDS), SARS, and systemic sclerosis.

In some embodiments, the compositions described herein are used to treat or alleviate a symptom of ARDS associated with a coronavirus infection and in particular COVID-19.

The compositions include formulations and compositions for administering the IL-6 antibodies by aerosolization. The disclosure also includes combination use of an anti-IL-6R mAb with dactinomycin and/or or a CD-3 mAb that produce a synergistic effect that reduces symptoms associated with pulmonary inflammatory diseases, for example, CRS, ARDS, systemic sclerosis, and a coronavirus disease, such as COVID-19, SARS, or MERS.

The antibody binds IL-6R in soluble form, or membrane bound (i.e., when expressed on a cell surface). The antibody is e.g., a fully human antibody. The antibodies used in the compositions and methods of the disclosure are collectively referred to herein as “IL-6Rc” or “huIL-6Rc” or “IL-6” or “IL-6R” antibodies.

Dactinomycin, also known as actinomycin D, is a crystalline antibiotic composed of a phenoxazone chromophore and two cyclic peptide chains obtained as a product of fermentation by Streptomyces parvulus.

Preferably, the dactinomycin is encapsulated in a nanoparticle, which has been demonstrated to reduce toxicities associated with free dactinomycin. The dactinomycin is encapsulated in nanoparticles of PLA-mPEG. The nanoparticles have an average size of about 100 nm to about 200 nm. (See, WO 2018/053052, the contents of which are hereby incorporated by reference in its entirety)

The exact mechanism of COVID-19 pathogenesis still remains to be discovered, it is suggested that the clinical manifestation of severe COVID-19 infection is also characterized by an over-exuberant immune response with lung lymphomononuclear cell infiltration and proliferation that may account for tissue damage more than the direct effect of viral replication.

Excessive production of pro-inflammatory cytokines such as IL-6 and TNF-α are the major factors contributing to progression of disease, lung tissue damage and eventually respiratory failures. Therefore a combination of anti-IL-6R mAb either with anti-TNFα mAb or with drugs that strengthen immune system (immune boosters), are immunomodulatory and/or suppresses hyperactive immune responses (such as an CD3 mAB) may be used as an immediate treatment for patients infected with coronavirus such as COVID-19 and other related family of viruses such as SARS and MERS.

Strikingly, these pulmonary manifestations are due to intense local inflammatory responses and immune dysregulation underlying the profound pulmonary pathology. It is well established that macrophage and dendritic cells not only are critically involved in mediating acute inflammatory responses but also are central players in bridging innate and adaptive immunity against microbial infections. The transient decrease in the circulating CD4 and CD8 T-cell subsets has been reported to positively correlate with the adverse outcome of coronavirus infection, such as that caused by SARS-CoV, SARS-CoV-2, and MERS-CoV.

Dactinomycin binds to DNA and inhibits RNA synthesis (transcription), with chain elongation more sensitive than initiation, termination, or release. As a result of impaired mRNA production dactinomycin has been shown to have anti-viral effects.

Hence, a combination of anti-IL-6R (anti-IL-6 receptor) mAb and dactinomycin either with anti-TNFα mAb or with drugs that strengthen immune system (immune boosters), are immunomodulatory and/or suppresses hyperactive immune responses (such as an CD3 mAB) may be used as an immediate treatment for patients infected with coronavirus such as COVID-19 and other related family of viruses such as SARS and MERS. Dactinomycin may be a nanoparticle formulation.

### IL-6/IL-6 Receptor Antibodies

Exemplary IL-6/IL-6 receptor (IL-6R) antibodies useful in the compositions and methods of the disclosure include, for example, Actemra® (tocilizumab), or Kevzera® (sarilumab) and anti-IL-6 mAbs.

Other, IL-6R antibodies include the 39B9 VL1 antibody, the 39B9 VL5 antibody, the 12A antibody, and the 5C antibody described in WO/2009/140348, the contents of which are hereby incorporated by reference in its entirety. These antibodies show specificity for human IL-6R and/or both IL-6R and IL-6Rc and they have been shown to inhibit the functional activity of IL-6Rc (i.e., binding to gp130 to induce the signaling cascade) in vitro.

In some embodiments, the anti-6Rc antibody comprises a light chain and a heavy chain sequence. In some embodiments, the anti-6R antibody light chain sequence is SEQ ID NO: 53. In some embodiments, the anti-6Rc antibody heavy chain sequence is SEQ ID NO: 52. In some embodiments, the anti-6Rc antibody comprises SEQ ID NO: 53 and SEQ ID NO: 52.

The 39B9 VL1 and 39B9 VL5 antibodies share a common heavy chain variable region (SEQ ID NO:2) encoded by the nucleic acid sequence shown in SEQ ID NO: 1. The 39B9 VL1 antibody includes a light chain variable region (SEQ ID NO: 4) encoded by the nucleic acid sequence shown in SEQ ID NO: 3. The 39B9 VL5 antibody includes a light chain variable region (SEQ ID NO: 6) encoded by the nucleic acid sequence shown in SEQ ID NO: 5. The 12A antibody includes a heavy chain variable region (SEQ ID NO:8) encoded by the nucleic acid sequence shown in SEQ ID NO: 7. The 12A antibody includes a light chain variable region (SEQ ID NO: 10) encoded by the nucleic acid sequence shown in SEQ ID NO: 9. The 5C antibody includes a heavy chain variable region (SEQ ID NO: 12) encoded by the nucleic acid sequence shown in SEQ ID NO: 11. The 5C antibody includes a light chain variable region (SEQ ID NO: 14) encoded by the nucleic acid sequence shown in SEQ ID NO: 13.

TABLE 1
Illustrative IL-6/IL-6R Antibody Sequences

| Name | SEQ ID NO. | Sequence |
|---|---|---|
| 39B9 VL1-VH nucleic | 1 | CAGGTGCAGCTGGTGCAGTCTGGGGCTGAGGTGAAGAAGCCTGGG |
| acid sequence | | TCCTCGGTGAAGGTCTCCTGCAAGGCTTCTGGAGGCACCTTCAGC |
| | | AGCTATGCTATCAGCTGGGTGCGCCAGGCCCCTGGACAAGGGCTT |
| | | GAGTGGATGGGAGGGATCATCCCTCTCTTTGATACAACAAAGTAC |
| | | GCACAGCAGTTCCAGGGCAGAGTCACGATTACCGCGGACGAATCC |
| | | ACGAGCACAGCCTACATGGAGCTGAGCAGCCTGAGATCTGAGGAC |
| | | ACGGCCGTATTTTACTGTGCGAGAGATCGGGATATTTTGACTGAT |
| | | TATTATCCCATGGGCGGTATGGACGTCTGGGGCCAAGGGACCACG |
| | | GTCACCGTCTCCTCA |
| 39B9 VL1-VH amino | 2 | QVQLVQSGAEVKKPGSSVKVSCKASGGTFSSYAISWVRQAPGQGL |
| acid sequence | | EWMGGIIPLFDTTKYAQQFQGRVTITADESTSTAYMELSSLRSED |
| | | TAVFYCARDRDILTDYYPMGGMDVWGQGTTVTVSS |
| 39B9 VL1-VL nucleic | 3 | GCCATCCAGTTGACCCAGTCTCCATCCTCCCTGTCTGCATCTGTA |
| acid sequence | | GGAGACAGAGTCACCATCACTTGCCGGGCAAGTCAGGGCATTAGC |
| | | AGTGTTTTAGCCTGGTATCAGCAGAAACCAGGGAAAGCTCCTAAG |
| | | CTCCTGATCTATGATGCCTCCAGTTTGGAAAGTGGGGTCCCATCA |
| | | AGGTTCAGCGGCAGTGGATCTGGGACAGATTTCACTCTCACCATC |
| | | AGCAGCCTGCAGCCTGAAGATTTTGCAACTTATTACTGTCAACAG |
| | | TCTAATAGTTACCCGCTCACTTTCGGCGGAGGGACCAAGGTGGAG |
| | | ATCAAACGT |
| 39B9 VL1-VL amino | 4 | AIQLTQSPSSLSASVGDRVTITCRASQGISSVLAWYQQKPGKAPK |
| acid sequence | | LLIYDASSLESGVPSRFSGSGSGTDFTLTISSLQPEDFATYYCQQ |
| | | SNSYPLTFGGGTKVEIKR |
| 39B9 VL5-VL nucleic | 5 | GACATCCTGATGACCCAGTCTCCATCCTCCCTGTCTGCATCTGTA |
| acid sequence | | GGAGACAGAGTCACCATCACTTGTCGGGCGAGTCAGGATATTAGC |
| | | AGCTGGTTAGCCTGGTATCAGCAGAAACCAGGGAAAGCTCCTAAG |
| | | CTCCTGATCTATGATGCCTCCAGTTTGGAAAGTGGGGTCCCATCA |
| | | AGGTTCAGCGGCAGTGGATCTGGGACAGATTTCACTCTCACCATC |
| | | AGCAGCCTGCAGCCTGAAGATTTTGCAACTTATTACTGTCAACAG |
| | | TCTAATAGTTACCCGCTCACTTTCGGCGGAGGGACCAAGGTGGAG |
| | | ATCAAACGA |
| 39B9 VL5-VL amino | 6 | DILMTQSPSSLSASVGDRVTITCRASQDISSWLAWYQQKPGKAPK |
| acid sequence | | LLIYDASSLESGVPSRFSGSGSGTDFTLTISSLQPEDFATYYCQQ |
| | | SNSYPLTFGGGTKVEIKR |
| 12A VH nucleic acid | 7 | CAGGTGCAGCTGGTGGAGTCTTGGGGAGGCGTGGTCCAGCCTGGG |
| sequence | | AGGTCCCTGAGACTCTCCTGTGCAGCGTCTGGATTCACCTTCAGT |
| | | AACTATGACATGTACTGGGTCCGCCAGGCTCCAGGCAAGGGGCTG |
| | | GAGTGGGTGGCAGTTATATTAGATGATGGAAATAATAATTACTAC |
| | | GCAGACTCCGTGAAGGGCCGATTCACCATCTCCAGAGACAATTCC |
| | | AAGAAAAAGGTGTATCTGCAAATGAATAGCCTGAGAGCTGAGGAC |
| | | ACGGCTGTGTATTACTGTGTGAGAGCGTCCCCTAACTGGGGTCTT |
| | | CTTGACTTCTGGGGCCAGGGAACCCTGGTCACCGTCTCGAGT |
| 12A VH amino acid | 8 | QVQLVESWGGVVQPGRSLRLSCAASGFTFSNYDMYWVRQAPGKGL |
| sequence | | EWVAVILDDGNNNYYADSVKGRFTISRDNSKKKVYLQMNSLRAED |
| | | TAVYYCVRASPNWGLLDFWGQGTLVTVSS |
| 12A VL nucleic acid | 9 | GAAATTGTGTTGACACAGTCTCCATCCTCACTGTCTGCATCTGTA |
| sequence | | GGAGACAGAGTCACCATCACTTGTCGGGCGAGTCAGGGTATTAGC |
| | | AGCTGGTTAGCCTGGTATCAGCAGAAACCAGGGAAAGCTCCTAAG |
| | | CTCCTGATCTATGATGCCTCCAGTTTGGAAAGTGGGGTCCCATCA |
| | | AGGTTCAGCGGCAGTGGATCTGGGACAGATTTCACTCTCACCATC |
| | | AGCAGCCTGCAGCCTGAAGATTTTGCAACTTATTACTGTCAACAG |
| | | TTTAATAGTTACCCGATCACCTTCGGCCAAGGGACACGACTGGAG |
| | | ATTAAACGT |
| 12A VL amino acid | 10 | EIVLTQSPSSLSASVGDRVTITCRASQGISSWLAWYQQKPGKAPK |
| sequence | | LLIYDASSLESGVPSRFSGSGSGTDFTLTISSLQPEDFATYYCQQ |
| | | FNSYPITFGQGTRLEIKR |
| 5C VH nucleic acid | 11 | CAGGTGCAGCTGGTGCAGTCTGGGGGAGGCGTGGTCCAGCCTGGG |
| sequence | | AGGTCCCTGAGACTCTCCTGTGCAGCCTCTGGATTCATCTTCAGT |
| | | AGCTATGACATGTACTGGGTCCGCCAGGCTCCAGGCAAGGGGCTG |
| | | GAGTGGGTGGCAGTTATATTATATGATGGAAATAATAAATACTAC |
| | | GCAGACTCCGTGAAGGGCCGATTCACCATCTCCAGAGACAATTCC |
| | | AAGAACACGGTGTATCTGCAAATGAACAGCCTGAGAGCTGAGGAC |
| | | ACGGCTGTGTATTACTGTGTGAGAGCGTCCCCTAACTGGGGTCTT |
| | | TTTGACTTCTGGGGCCAGGGAACCCTGGTCACCGTCTCGAGT |
| 5C VH amino acid | 12 | QVQLVQSGGGVVQPGRSLRLSCAASGFIFSSYDMYWVRQAPGKGL |
| sequence | | EWVAVILYDGNNKYYADSVKGRFTISRDNSKNTVYLQMNSLRAED |
| | | TAVYYCVRASPNWGLFDFWGQGTLVTVSS |
| 5C VL nucleic acid | 13 | GACATCCAGATGACCCAGTCTCCATCCTCCCTGTCTGCATCTGTA |
| sequence | | GGAGACAGAGTCACCATCACTTGCCGGGCAAGTCAGGGCATTAGC |
| | | AGTGATTTAGCCTGGTATCAGCAGAAACCAGGGAAAGCTCCTAAG |
| | | CTCCTGATGTATGATGCCTCCAGTTTGGAAAGTGGGGTCCCATCA |
| | | AGGTTCAGCGGCAGTGGATCTGGGACAGATTTCACTCTCACCATC |
| | | AGCAGCCTGCAGCCTGAAGATTTTGCAACTTATTACTGTCAACAG |
| | | TTTAATAGTTACCCGATCACCTTCGGCCAAGGGACACGACTGGAG |
| | | ATTAAACGT |
| 5C VL amino acid | 14 | DIQMTQSPSSLSASVGDRVTITCRASQGISSDLAWYQQKPGKAPK |
| sequence | | LLMYDASSLESGVPSRFSGSGSGTDFTLTISSLOPEDFATYYCQQ |
| | | FNSYPITFGQGTRLEIKR |

huIL-6R antibodies of the disclosure additionally comprise, for example, the heavy chain complementarity determining regions (VH CDRs) shown below in Table 2, the light chain complementarity determining regions (VL CDRs) shown in Table 3, and combinations thereof.

TABLE 2
VH CDR sequences from antibody clones that bind and neutralize IL-6R
biological activity

| Clone Name | VH CDR1 | VH CDR2 | VH CDR3 |
|---|---|---|---|
| 39B9 | SYAIS | GIIPLFDTTKYAQQFQG | CARDRDILTDYYPMGGMDV |
| | (SEQ ID NO: 15) | (SEQ ID NO: 16) | (SEQ ID NO: 17) |
| 12A | NYDMY | VILDDGNNNYYADSVKG | CVRASPNWGLLDF |
| | (SEQ ID NO: 18) | (SEQ ID NO: 19) | (SEQ ID NO: 20) |
| 5C | SYDMY | VILYDGNNKYYADSVKG | CVRASPNWGLFDF |
| | (SEQ ID NO: 21) | (SEQ ID NO: 22) | (SEQ ID NO: 23) |

TABLE 3
VL CDR sequences from antibody clones that bind and neutralize IL-6Rc

| Clone Name | VL CDR1 | VL CDR2 | VL CDR3 |
|---|---|---|---|
| 39B9 VL1 | RASQGISSVLA | DASSLES | QQSNSYPLT |
| | (SEQ ID NO: 24) | (SEQ ID NO: 25) | (SEQ ID NO: 26) |
| 39B9 VL5 | RASQDISSWLA | DASSLES | QQSNSYPLT |
| | (SEQ ID NO: 27) | (SEQ ID NO: 25) | (SEQ ID NO: 26) |
| 12A | RASQGISSWLA | DASSLES | QQSNSYPIT |
| | (SEQ ID NO: 28) | (SEQ ID NO: 25) | (SEQ ID NO: 29) |
| 5C | RASQGISSVDA | DASSLES | QQSNSYPIT |
| | (SEQ ID NO: 30) | (SEQ ID NO: 25) | (SEQ ID NO: 29) |

The huIL-6R antibodies of the disclosure serve to modulate, block, inhibit, reduce, antagonize, neutralize or otherwise interfere with the functional activity of IL-6Rc. Functional activities of IL-6Rc include for example, intracellular signaling via activation of the JAK/STAT pathway and activation of the MAPK cascade, acute phase protein production, antibody production and cellular differentiation and/or proliferation. For example, the huIL-6R antibodies completely or partially inhibit IL-6Rc functional activity by partially or completely modulating, blocking, inhibiting, reducing antagonizing, neutralizing, or otherwise interfering with the binding of IL-6Rc to the signal-transducing receptor component gp130.

The huIL-6R antibodies are considered to completely modulate, block, inhibit, reduce, antagonize, neutralize or otherwise interfere with IL-6Rc functional activity when the level of IL-6Rc functional activity in the presence of the huIL-6R antibody is decreased by at least 95% o, e.g., by 9600, 97%, 98% o, 9900 or 100% o as compared to the level of IL-6Rc functional activity in the absence of binding with a huIL-6R antibody described herein. The huIL-6R antibodies are considered to partially modulate, block, inhibit, reduce, antagonize, neutralize or otherwise interfere with IL-6Rc functional activity when the level of IL-6Rc activity in the presence of the huIL-6R antibody is decreased by less than 95%, e.g., 10%, 20%, 25%, 30%, 40%, 50%, 60%, 75%, 80%, 85% or 90% as compared to the level of IL-6Rc activity in the absence of binding with a huIL-6R antibody described herein.

Variants of huIL-6R Antibodies

Variants of the huIL-6R antibodies are made using any of a variety of art-recognized techniques. For example, variant huIL-6R antibodies include antibodies having one or more amino acid modifications, such as, for example, an amino acid substitution, at position within the antibody sequence.

Preferred locations for amino acid substitutions are shown as bold, underlined residues below in Table 4. The amino acid residues in bold/underline can be replaced with any amino acid residue. In preferred embodiments, the amino acid residues in bold/underline are replaced with the amino acid residues shown below in Table 4. In these embodiments, the antibody comprises (i) the consensus amino acid sequence QQSXSYPLT (SEQ ID NO: 31) in the light chain complementarity determining region 3 (CDR3), where X is N or Q; (ii) the consensus amino acid sequence GIIPX1FX2TTKYAQX3FQG (SEQ ID NO: 32) in the heavy chain complementarity determining region 2 (CDR2), where X1 is L or A, X2 is D or E, and X3 is Q or K; (iii) the consensus amino acid sequence DRDILTDYYPXGGMDV (SEQ ID NO: 33) in the heavy chain complementarity determining region 3 (CDR3), where X is M or L; and (iv) the consensus amino acid sequence TAVXYCAR (SEQ ID NO: 34) in the framework region 3 (FRW3), where X is F or Y.

The NI-1201-wild type (NI-1201-WT) antibody listed in Table 4 comprises the amino acid sequence QQSNSYPLT (SEQ ID NO: 26) in the light chain CDR3 region, the amino acid sequence GIIPLFDTTKYAQQFQG (SEQ ID NO: 16) in the heavy chain CDR2 region, the amino acid sequence DRDILTDYYPMGGMDV (SEQ ID NO: 35) in the heavy chain CDR3 region, and the amino acid sequence TAVFYCAR (SEQ ID NO: 36) in the FRW3 region.

The NI-1201-A antibody listed in Table 4 comprises the amino acid sequence QQSNSYPLT (SEQ ID NO: 26) in the light chain CDR3 region, the amino acid sequence GIIPLFDTTKYAQKFQG (SEQ ID NO: 37) in the heavy chain CDR2 region, the amino acid sequence DRDILTDYYPMGGMDV (SEQ ID NO: 35) in the heavy chain CDR3 region, and the amino acid sequence TAVYYCAR (SEQ ID NO: 38) in the FRW3 region.

The NI-1201-B antibody listed in Table 4 comprises the amino acid sequence QQSNSYPLT (SEQ ID NO: 26) in the light chain CDR3 region, the amino acid sequence GIIPLFDTTKYAQKFQG (SEQ ID NO: 37) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-C antibody listed in Table 4 comprises the amino acid sequence QQSNSYPLT (SEQ ID NO: 26) in the light chain CDR3 region, the amino acid sequence GIIPAFETTKYAQKFQG (SEQ ID NO: 40) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-D antibody listed in Table 4 comprises the amino acid sequence QQSQSYPLT (SEQ ID NO: 41) in the light chain CDR3 region, the amino acid sequence GIIPAFETTKYAQKFQG (SEQ ID NO: 40) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-E antibody listed in Table 4 comprises the amino acid sequence QQSQSYPLT (SEQ ID NO: 41) in the light chain CDR3 region, the amino acid sequence GIIPLFDTTKYAQKFQG (SEQ ID NO: 37) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-F antibody listed in Table 4 comprises the amino acid sequence QQSNSYPLT (SEQ ID NO: 26) in the light chain CDR3 region, the amino acid sequence GIIPAFDTTKYAQKFQG (SEQ ID NO: 42) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-G antibody listed in Table 4 comprises the amino acid sequence QQSQSYPLT (SEQ ID NO: 41) in the light chain CDR3 region, the amino acid sequence GIIPAFDTTKYAQKFQG (SEQ ID NO: 42) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

TABLE 4
NI-1201 Leads

| | Light Chain | | | |
|---|---|---|---|---|
| | CDR3 | Heavy Chain CDR2 | Heavy Chain CDR3 | FRW 3 |
| NI- | QQSNSYPLT | GIIPLFDTTKYAQQFOG | DRDILTDYYPMGGMDV | TAVFYCAR |
| 1201-WT | (SEQ ID | (SEQ ID NO: 16) | (SEQ ID NO: 35) | (SEQ ID NO: 36) |
| | NO: 26) | | | |
| NI- | QQSNSYPLT | GIIPLFDTTKYAQKFQG | DRDILTDYYPMGGMDV | TAVYYCAR |
| 1201-A | (SEQ ID | (SEQ ID NO: 37) | (SEQ ID NO: 35) | (SEQ ID NO: 38) |
| | NO: 26) | | | |
| NI- | QQSNSYPLT | GIIPLFDTTKYAQKFQG | DRDILTDYYPLGGMDV | TAVYYCAR |
| 1201-B | (SEQ ID | (SEQ ID NO: 37) | (SEQ ID NO: 39) | (SEQ ID NO: 38) |
| | NO: 26) | | | |
| NI- | QQSNSYPLT | GIIPAFETTKYAQKFQG | DRDILTDYYPLGGMDV | TAVYYCAR |
| 1201-C | (SEQ ID | (SEQ ID NO: 40) | (SEQ ID NO: 39) | (SEQ ID NO: 38) |
| | NO: 26) | | | |
| NI- | QQSQSYPLT | GIIPAFETTKYAQKFQG | DRDILTDYYPLGGMDV | TAVYYCAR |
| 1201-D | (SEQ ID | (SEQ ID NO: 40) | (SEQ ID NO: 39) | (SEQ ID NO: 38) |
| | NO: 41) | | | |
| NI- | QQSQSYPLT | GIIPLFDTTKYAQKFQG | DRDILTDYYPLGGMDV | TAVYYCAR |
| 1201-E | (SEQ ID | (SEQ ID NO: 37) | (SEQ ID NO: 39) | (SEQ ID NO: 38) |
| | NO: 41) | | | |
| NI- | QQSNSYPLT | GIIPAFDTTKYAQKFQG | DRDILTDYYPLGGMDV | TAVYYCAR |
| 1201-F | (SEQ ID | (SEQ ID NO: 42) | (SEQ ID NO: 39) | (SEQ ID NO: 38) |
| | NO: 26) | | | |
| NI- | QQSQSYPLT | GIIPAFDTTKYAQKFQG | DRDILTDYYPLGGMDV | TAVYYCAR |
| 1201-G | (SEQ ID | (SEQ ID NO: 42) | (SEQ ID NO: 39) | (SEQ ID NO: 38) |
| | NO: 41) | | | |

### Anti-CD3 Antibodies

In some embodiments, the disclosure provides methods for treatment of coronavirus infection and associated symptoms by administering an anti-CD3 antibody in combination with an anti-IL-6R antibody. In some embodiments, the anti-CD3 antibody is administered in combination of an with an anti-IL-6R antibody and dactinomycin.

The anti-CD3 antibodies can be any antibodies specific for CD3. The anti-CD3 antibody can be a polyclonal, monoclonal, recombinant, e.g., a chimeric, de-immunized or humanized, fully human, non-human, e.g., murine, single chain antibody or single domain antibody. In some embodiments the antibody has effector function and can fix complement. In some embodiments, the antibody has reduced or no ability to bind an Fc receptor. For example, the anti-CD3 antibody can be an isotype or subtype, fragment or other mutant, which does not support binding to an Fc receptor, e.g., it has a mutagenized or deleted Fc receptor binding region. The antibody can be coupled to a toxin or imaging agent.

A number of anti-CD3 antibodies are known, including but not limited to OKT3 (muromonab/Orthoclone OKT3™, Ortho Biotech, Raritan, N.J.; U.S. Pat. No. 4,361,549); hOKT3(1 (Herold et al., N.E.J.M. 346(22):1692-1698 (2002); HuM291 (Nuvion™, Protein Design Labs, Fremont, Calif.); gOKT3-5 (Alegre et al., J. Immunol. 148(11):3461-8 (1992); 1F4 (Tanaka et al., J. Immunol. 142:2791-2795 (1989)); G4.18 (Nicolls et al., Transplantation 55:459-468 (1993)); 145-2C11 (Davignon et al., J. Immunol. 141(6):1848-54 (1988)); and as described in Frenken et al., Transplantation 51(4):881-7 (1991); U.S. Pat. Nos. 6,491,9116, 6,406,696, and 6,143,297).

Methods for making such antibodies are also known. A full-length CD3 protein or antigenic peptide fragment of CD3 can be used as an immunogen, or can be used to identify anti-CD3 antibodies made with other immunogens, e.g., cells, membrane preparations, and the like, e.g., E rosette positive purified normal human peripheral T cells, as described in U.S. Pat. Nos. 4,361,549 and 4,654,210. The anti-CD3 antibody can bind an epitope on any domain or region on CD3.

Chimeric, humanized, de-immunized, or completely human antibodies are desirable for applications which include repeated administration, e.g., therapeutic treatment of human subjects.

Chimeric antibodies contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and Oi, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The compositions and methods of the disclosure also include partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The anti-CD3 antibody can also be a single chain antibody. A single-chain antibody (scFV) can be engineered (see, for example, Colcher et al., Ann. N. Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target CD3 protein. In some embodiments, the antibody is monovalent, e.g., as described in Abbs et al., Ther. Immunol. 1(6):325-31 (1994), incorporated herein by reference.

Exemplary anti-CD3 antibodies, comprise a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47).

In some embodiments, the anti-CD3 antibody comprises a variable heavy chain amino acid sequence comprising QVQLVESGGGVVQPGRSLRLSCAASGFKFSGYGMHWVRQAPGKGLEWVAVIWYDGS KKYYVDSVKGRFTISRDNSKNTLYLQMNSLRAEDTAVYYCARQMGYWHFDLWGRGT LVTVSS (SEQ ID NO: 48) and a variable light chain amino acid sequence comprising EIVLTQSPATLSLSPGERATLSCRASQSVSSYLAWYQQKPGQAPRLLIYDASNRATGIPA RFSGSGSGTDFTLTISSLEPEDFAVYYCQQRSNWPPLTFGGGTKVEIK (SEQ ID NO: 49).

Preferably, the anti-CD3 antibody comprises a heavy chain amino acid sequence comprising: QVQLVESGGGVVQPGRSLRLSCAASGFKFSGYGMHWVRQAPGKGLEWVAVIWYDGS KKYYVDSVKGRFTISRDNSKNTLYLQMNSLRAEDTAVYYCARQMGYWHFDLWGRGT LVTVSSASTKGPSVFPLAPSSKSTSGGTAALGCLVKDYFPEPVTVSWNSGALTSGVHTFP AVLQSSGLYSLSSVVTVPSSSLGTQTYICNVNHKPSNTKVDKRVEPKSCDKTHTCPPCPA PEAEGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFL YSKLTVDKSRWQQGNVFSCSVMHEALHNHYTQKSLSLSPGK (SEQ ID NO: 50) and a light chain amino acid sequence comprising: EIVLTQSPATLSLSPGERATLSCRASQSVSSYLAWYQQKPGQAPRLLIYDASNRATGIPA RFSGSGSGTDFTLTISSLEPEDFAVYYCQQRSNWPPLTFGGGTKVEIKRTVAAPSVFIFPPS DEQLKSGTASVVCLLNNFYPREAKVQWKVDNALQSGNSQESVTEQDSKDSTYSLSSTL TLSKADYEKHKVYACEVTHQGLSSPVTKSFNRGEC (SEQ ID NO: 51). This anti-CD3 antibody is referred to herein as NI-0401, Foralumab, or 28F11-AE. (See e.g., Dean Y, Depis F, Kosco-Vilbois M. “Combination therapies in the context of anti-CD3 antibodies for the treatment of autoimmune diseases.” Swiss Med Wkly. (2012) (the contents of which are hereby incorporated by reference in its entirety).

In some embodiments, the anti-CD3 antibody is a fully human antibody or a humanized antibody. In some embodiments, the anti-CD3 antibody formulation includes a full length anti-CD3 antibody. In alternative embodiments, the anti-CD3 antibody formulation includes an antibody fragment that specifically binds CD3. In some embodiments, the anti-CD3 antibody formulation includes a combination of full-length anti-CD3 antibodies and antigen binding fragments that specifically bind CD3.

In some embodiments, the antibody or antigen-binding fragment thereof that binds CD3 is a monoclonal antibody, domain antibody, single chain, Fab fragment, a F(ab′)2 fragment, a scFv, a scAb, a dAb, a single domain heavy chain antibody, or a single domain light chain antibody. In some embodiments, such an antibody or antigen-binding fragment thereof that binds CD3 is a mouse, other rodent, chimeric, humanized or fully human monoclonal antibody.

Optionally, the anti-CD3 antibody or antigen binding fragment thereof used in the formulations of the disclosure includes at least one amino acid mutation. Typically, the mutation is in the constant region. The mutation results in an antibody that has an altered effector function. An effector function of an antibody is altered by altering, i.e., enhancing or reducing, the affinity of the antibody for an effector molecule such as an Fc receptor or a complement component. For example, the mutation results in an antibody that is capable of reducing cytokine release from a T-cell. For example, the mutation is in the heavy chain at amino acid residue 234, 235, 265, or 297 or combinations thereof. Preferably, the mutation results in an alanine residue at either position 234, 235, 265 or 297, or a glutamate residue at position 235, or a combination thereof.

Preferably, the anti-CD3 antibody provided herein contains one or more mutations that prevent heavy chain constant region-mediated release of one or more cytokine(s) in vivo.

In some embodiments, the anti-CD3 antibody or antigen binding fragment thereof used in the formulations of the disclosure is a fully human antibody. The fully human CD3 antibodies used herein include, for example, a L234A and L235E mutation in the Fc region, such that cytokine release upon exposure to the anti-CD3 antibody is significantly reduced or eliminated. The L234A and L235E mutation in the Fc region of the anti-CD3 antibodies provided herein reduces or eliminates cytokine release when the anti-CD3 antibodies are exposed to human leukocytes, whereas the mutations described below maintain significant cytokine release capacity. For example, a significant reduction in cytokine release is defined by comparing the release of cytokines upon exposure to the anti-CD3 antibody having an L234A and L235E mutation in the Fc region to level of cytokine release upon exposure to another anti-CD3 antibody having one or more of the mutations described below. Other mutations in the Fc region include, for example, L234A and L235A, L235E, N297A, D265A, or combinations thereof.

### Dactinomycin Nanoparticles

Dactinomycin may also be referred to as 2-amino-N,N′-bis(hexadecahydro-2,5,9-trimethyl-6,13-bis(1-methylethyl)-1,4,7,11,14-pentaoxo-1H-pyrrolo(2,1-I)(1,4,7,10,13)oxatetra-azacyclohexadecin-10-yl)-4,6-dimethyl-3-oxo-3H-phenoxazine-1,9-dicarboxamide, ActD, actinomycin C1, actinomycin D; actinomycin iv, dactinomicina, dactomycin, dactinomycine, dactinomycinum, or meractinomycin. Dactinomycin belongs to the class of organic compounds known as cyclic depsipeptides. Cyclic depsipeptides include natural and/or non-natural (i.e., synthetic) compounds having sequences of amino and hydroxy carboxylic acid residues (usually α-amino and α-hydroxy acids) connected in a ring. Amino and hydroxy carboxylic acid residues within dactinomycin may alternate in a repeating pattern.

Dactinomycin is a polypeptide antibiotic composed of two cyclic peptides attached to a phenoxazine that is derived from Streptomyces parvullus. Dactinomycin binds to DNA and inhibits RNA synthesis (transcription) by specifically interfering with chain elongation of mRNA transcripts. Dactinomycin binds strongly but reversibly to DNA molecules. As a result of impaired mRNA production, protein synthesis, ribosome biogenesis and cell division decline after dactinomycin therapy. Because dactinomycin inhibits mRNA production and protein synthesis, it is hypothesized that dactinomycin inhibits coronavirus production in the host cell.

Dactinomycin is extremely corrosive and may produce many adverse side effects, for example, tissue necrosis and mucositis may occur following extravasation days to weeks after administration at the infusion site. Mucositis, i.e., extremely painful blistering, inflammation, and ulceration of the mucous membranes lining the gastrointestinal tract. Mucositis that is adverse of debilitating to the degree that subjects being treated with dactinomycin may not able to continue and complete their treatment.

Most of the toxicities associated with dactonomycin is thought to be due to the very high levels of the drug in the first few hours after dosing and may be mitigated by administering dactinomycin doses in the range of 10 to 20 μg/kg/day in a controlled release manner per any of the dactinomycin nanoparticle compositions described herein while still maintaining efficacy for inhibiting mRNA synthesis.

A dactinomycin composition of the disclosure can modulate the activity of a molecular target (e.g., a ribosome). Modulating refers to stimulating or inhibiting an activity of a molecular target. Preferably, a dactinomycin composition of the disclosure modulates the activity of a molecular target if it stimulates or inhibits the activity of the molecular target by at least 2-fold relative to the activity of the molecular target under the same conditions but lacking only the presence of said compound. More preferably, a dactinomycin composition of the disclosure modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo.

The present disclosure provides compositions comprising a therapeutically effective amount of dactinomycin encapsulated in nanoparticles comprising one or more polymers. The polymers include, without limitation, poly(lactic acid) (PLA), poly(butyric acid), poly(valeric acid), poly(caprolactone) (PCL), poly(hydroxybutyrate), poly(lactide-co-caprolactone), poly(lactide-co-glycolide) (PLGA), polymethylcyanoacrylate, and polyanhydrides, poly(ortho)esters, or polyurethanes thereof, wherein the polymer optionally further comprises polyethylene glycol (PEG) or polyethylene glycol methyl ether (mPEG) and/or any combination thereof.

The present disclosure provides compositions comprising blends, i.e. mixtures of one more or polymers selected from poly(lactic acid) (PLA), poly(butyric acid), poly(valeric acid), poly(caprolactone) (PCL), poly(hydroxybutyrate), poly(lactide-co-caprolactone), poly(lactide-co-glycolide) (PLGA), polymethylcyanoacrylate, and polyanhydrides, poly(ortho)esters, or polyurethanes thereof.

The present disclosure also provides compositions comprising blends, i.e. mixtures of one more or polymers selected from poly(lactic acid) (PLA), poly(butyric acid), poly(valeric acid), poly(caprolactone) (PCL), poly(hydroxybutyrate), poly(lactide-co-caprolactone), poly(lactide-co-glycolide) (PLGA), polymethylcyanoacrylate, and polyanhydrides, poly(ortho)esters, or polyurethanes thereof, wherein the polymer further comprises polyethylene glycol (PEG), polyethylene glycol methyl ether (mPEG), and/or a combination thereof.

Those skilled in the art will recognize the that release characteristics and/or kinetics can be altered (e.g., increased or decreased) by selecting various combinations of these polymers. Determination of the appropriate polymer combinations to achieve the desired release rates is within the routine level of skill in the art.

A nanoparticle, as used herein, refers to a particle between about 1 and about 500 nanometers (nm) in size. For example, in one embodiment, between about 100 nm and about 200 nm.

In one embodiment, compositions of the disclosure include those compositions which comprise dactinomycin which is encapsulated in and/or associated with nanoparticles.

The present disclosure provides compositions comprising a therapeutically effective amount of dactinomycin encapsulated in nanoparticles comprising poly lactic acid (PLA) polymers. PLA is an amorphous (non-crystalline) biodegradable polymer that is typically a mixture of D and L enantiomeric forms of lactic acid. PLA is also available as crystalline polymer when synthesized using only a single enantiomer of lactic acid.

In one embodiment, the present disclosure provides for dactinomycin encapsulated in nanoparticles comprising PLA, where the PLA has a molecular weight ranging from about 10,000 to about 24,000 Da, and allows for a controlled release of dactinomycin.

In one embodiment, the molecular weight of the PLA is about 10,000 Da to about 18,000 Da.

In one embodiment, the molecular weight of the PLA is about 18,000 Da to about 24,000 Da.

In one embodiment, the molecular weight of the PLA is about 10,000 Da to about 14,000 Da, 11,000 Da to about 15,000 Da, 12,000 Da to about 16,000 Da, 13,000 Da to about 17,000 Da, 14,000 Da to about 18,000 Da, 15,000 Da to about 19,000 Da, 16,000 Da to about 20,000 Da, 17,000 Da to about 21,000 Da, 18,000 Da to about 22,000 Da, 19,000 Da to about 23,000 Da, or 20,000 Da to about 24,000 Da.

In one embodiment, the molecular weight of the PLA is about 10,000 Da, about 11,000 Da, about 12,000 Da, about 13,000 Da, about 14,000 Da, about 15,000 Da, about 16,000 Da, about 17,000 Da, about 18,000 Da, about 19,000 Da, about 20,000 Da, about 21,000 Da, about 22,000 Da, about 23,000 Da, about 24,000 Da.

The present disclosure provides compositions comprising a therapeutically effective amount of dactinomycin encapsulated in nanoparticles comprising PLA, where the PLA is ester-terminated.

The present disclosure provides compositions comprising a therapeutically effective amount of dactinomycin encapsulated in nanoparticles comprising PLA, where the PLA is acid-terminated form. Ester-terminated forms of PLA are more hydrophobic than acid-terminated forms of PLA, which are in turn more hydrophilic.

In one embodiment, compositions comprising a therapeutically effective amount of dactinomycin encapsulated in nanoparticles comprising ester-terminated PLAs degrade slower than dactinomycin encapsulated in nanoparticles comprising acid-terminated PLAs and would provide for a slower release of dactinomycin from nanoparticles.

The present disclosure provides compositions comprising a therapeutically effective amount of dactinomycin encapsulated in nanoparticles comprising poly (lactide-co-glycolide (PLGA) polymers:

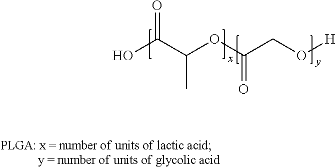

In aqueous environment, PLGA biodegrades by hydrolysis of its ester linkages.

The methyl side groups in PLA makes the polymer more hydrophobic than PGA. In one embodiment, lactide-rich PLGA copolymers are less hydrophilic, absorb less water and degrade more slowly compared to less lactide rich copolymers.

In one embodiment, higher molecular weight PLGAs degrade more slowly than lower molecular weight PLGAs.

In one embodiment, the present disclosure provides for dactinomycin encapsulated in nanoparticles comprising PLGA, where the PLGA has a molecular weight ranging from about 7,000 to about 54,000 Da, and allows for a controlled release of dactinomycin.

In one embodiment, the molecular weight of the PLGA is about 7,000 Da to about 17,000 Da, 17,000 Da to about 27,000 Da, 27,000 Da to about 37,000 Da, 37,000 Da to about 47,000 Da, or 47,000 Da to about 54,000 Da.

In one embodiment, the molecular weight of the PLGA is about 12,000 Da to about 22,000 Da, 22,000 Da to about 32,000 Da, 32,000 Da to about 42,000 Da, 42,000 Da to about 52,000 Da, or 44,000 Da to about 54,000 Da.

In one embodiment, the molecular weight of the PLGA is about 7,000 Da, about 8,000 Da, about 9,000 Da, about 10,000 Da, about 11,000 Da, about 12,000 Da, about 13,000 Da, about 14,000 Da, about 15,000 Da, about 16,000 Da, about 17,000 Da, about 18,000 Da, about 19,000 Da, about 20,000 Da, about 21,000 Da, about 22,000 Da, about 23,000 Da, about 24,000 Da, about 25,000 Da, about 26,000 Da, about 27,000 Da, about 28,000 Da, about 29,000 Da, about 30,000 Da, about 31,000 Da, about 32,000 Da, about 33,000 Da, about 34,000 Da, about 35,000 Da, about 36,000 Da, about 37,000 Da, about 38,000 Da, about 39,000 Da, about 40,000 Da, about 41,000 Da, about 42,000 Da, about 43,000 Da, about 44,000 Da, about 45,000 Da, about 46,000 Da, about 47,000 Da, about 48,000 Da, about 49,000 Da, about 50,000 Da, about 51,000 Da, about 52,000 Da, about 53,000 Da, or about 54,000 Da.

In one embodiment, the present disclosure provides for dactinomycin encapsulated in nanoparticles comprising PLGA, where the PLGA has a lactic acid:glycolic acid molar ratio of about 95:5, about 90:10, about 85:15, about 80:20, about. 75:25, about 70:30, about 65:35, about 60:40, about 55:45, about 50:50, about 45:55, about 40:60, about 35:65, about 30:70, about 25:75, about 20:80, about 15:85, about 10:90, or about 5:95.

In one embodiment, the present disclosure provides for dactinomycin encapsulated in nanoparticles comprising PLGA, where the PLGA has a lactic acid:glycolic acid molar ratio of about 50:50.

The present disclosure provides compositions comprising a therapeutically effective amount of dactinomycin encapsulated in nanoparticles comprising poly lactic acid (PLA) polymers, where the PLA polymers can be synthesized with polyethylene glycol (PEG) or polyethylene glycol methyl ether (mPEG) to prepare di-block copolymers (PLA-PEG and PLA-mPEG) and allow for a controlled release of dactinomycin.

In one embodiment, PLA-mPEG copolymer can be prepared according to the following scheme:

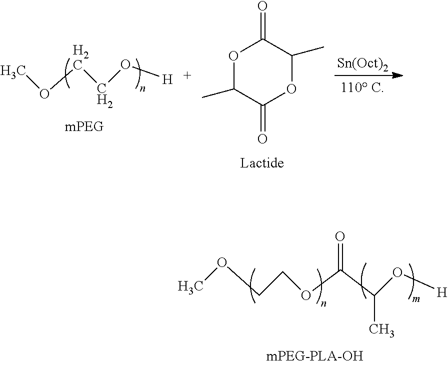

Hydrophilic PEG (and mPEG) moieties orient towards the surface of the nanoparticles and act as a barrier reducing interaction with foreign molecules by steric and hydrated repulsion. These compositions may have a longer half-life in systemic circulation compared to unmodified, nanoparticles, i.e., “non-PEG-ylated,” or “non-mPEG-ylated” nanoparticles.

In one embodiment, drug release from PLA-PEG and PLA-mPEG nanoparticles occurs through diffusion. In one embodiment, drug release PLA-PEG and PLA-mPEG nanoparticles occurs through polymer degradation. In one embodiment, drug release PLA-PEG and PLA-mPEG nanoparticles occurs through diffusion and polymer degradation.

The ratio of PEG or mPEG to PLGA may influence clearance of the nanoparticles from systemic circulation.

In one embodiment, the weight percent of PEG or mPEG in the PLA-PEG or PLA-mPEG di-block copolymers is about 1% by weight, about 2% by weight, about 3% by weight, about 4% by weight, about 5% by weight, about 6% by weight, about 7% by weight, about 8% by weight, about 9% by weight, about 10% by weight, about 11% by weight, about 12% by weight, about 13% by weight, about 14% by weight, about 15% by weight, about 16% by weight, about 17% by weight, about 18% by weight, about 19% by weight, about 20% by weight, about 21% by weight, about 22% by weight, about 23% by weight, about 24% by weight, about 25% by weight, about 26% by weight, about 27% by weight, about 28% by weight, about 29% by weight, about 30% by weight, about 31% by weight, about 32% by weight, about 33% by weight, about 34% by weight, about 35% by weight, about 36% by weight, about 37% by weight, about 38% by weight, about 39% by weight, about 40% by weight, about 41% by weight, about 42% by weight, about 43% by weight, about 44% by weight, about 45% by weight, about 46% by weight, about 47% by weight, about 48% by weight, about 49% by weight, about 50% by weight, about 51% by weight, about 52% by weight, about 53% by weight, about 54% by weight, about 55% by weight, about 56% by weight, about 57% by weight, about 58% by weight, about 59% by weight, about 60% by weight, about 61% by weight, about 62% by weight, about 63% by weight, about 64% by weight, about 65% by weight, about 66% by weight, about 67% by weight, about 68% by weight, about 69% by weight, about 70% by weight, about 71% by weight, about 72% by weight, about 73% by weight, about 74% by weight, about 75% by weight, about 76% by weight, about 77% by weight, about 78% by weight, about 79% by weight, about 80% by weight, about 81% by weight, about 82% by weight, about 83% by weight, about 84% by weight, about 85% by weight, about 86% by weight, about 87% by weight, about 88% by weight, about 89% by weight, about 90% by weight, about 91% by weight, about 92% by weight, about 93% by weight, about 94% by weight, about 95% by weight, about 96% by weight, about 97% by weight, about 98% by weight, or about 99% by weight.

In one embodiment, the weight percent of PEG or mPEG in the PLA-PEG or PLA-mPEG di-block copolymers is about 1% to 99%, 2% to 95%, 3% to 90%, 4% to 75%, 5% to 50%, 10% to 45%, 15% to 40%, 18% to 35%, or 20 to 30% by weight.

In one embodiment, the weight percent of PEG or mPEG in the PLA-PEG or PLA-mPEG di-block copolymers is about 25% by weight.

In one embodiment, the weight percent of mPEG in the PLA-mPEG di-block copolymer is about 25% by weight.

The present disclosure provides dactinomycin compositions comprising a therapeutically effective amount of dactinomycin encapsulated in nanoparticles comprising poly (lactide-co-glycolide (PLGA) polymers, where the PLGA polymers can be synthesized with polyethylene glycol (PEG) or polyethylene glycol methyl ether (mPEG) to prepare di-block copolymers (PLGA-PEG and PLGA-mPEG) and allow for a controlled release of dactinomycin. In one embodiment, PLGA-mPEG copolymer has the general structure illustrated in the scheme below:

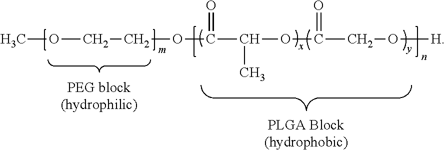

Hydrophilic PEG (and mPEG) moieties orient towards the surface of the nanoparticles and act as a barrier reducing interaction with foreign molecules by steric and hydrated repulsion. These compositions may have a longer half-life in systemic circulation compared to unmodified, nanoparticles, i.e., “non-PEG-ylated,” or “non-mPEG-ylated” nanoparticles.

In one embodiment, drug release from PLGA-PEG and PLGA-mPEG nanoparticles occurs through diffusion. In one embodiment, drug release PLGA-PEG and PLGA-mPEG nanoparticles occurs through polymer degradation. In one embodiment, drug release PLGA-PEG and PLGA-mPEG nanoparticles occurs through diffusion and polymer degradation.

The ratio of PEG or mPEG to PLGA may influence clearance of the nanoparticles from systemic circulation.

In one embodiment, the weight percent of PEG or mPEG in the PLGA-PEG or PLGA-mPEG di-block copolymers is about 1% by weight, about 2% by weight, about 3% by weight, about 4% by weight, about 5% by weight, about 6% by weight, about 7% by weight, about 8% by weight, about 9% by weight, about 10% by weight, about 11% by weight, about 12% by weight, about 13% by weight, about 14% by weight, about 15% by weight, about 16% by weight, about 17% by weight, about 18% by weight, about 19% by weight, about 20% by weight, about 21% by weight, about 22% by weight, about 23% by weight, about 24% by weight, about 25% by weight, about 26% by weight, about 27% by weight, about 28% by weight, about 29% by weight, about 30% by weight, about 31% by weight, about 32% by weight, about 33% by weight, about 34% by weight, about 35% by weight, about 36% by weight, about 37% by weight, about 38% by weight, about 39% by weight, about 40% by weight, about 41% by weight, about 42% by weight, about 43% by weight, about 44% by weight, about 45% by weight, about 46% by weight, about 47% by weight, about 48% by weight, about 49% by weight, about 50% by weight, about 51% by weight, about 52% by weight, about 53% by weight, about 54% by weight, about 55% by weight, about 56% by weight, about 57% by weight, about 58% by weight, about 59% by weight, about 60% by weight, about 61% by weight, about 62% by weight, about 63% by weight, about 64% by weight, about 65% by weight, about 66% by weight, about 67% by weight, about 68% by weight, about 69% by weight, about 70% by weight, about 71% by weight, about 72% by weight, about 73% by weight, about 74% by weight, about 75% by weight, about 76% by weight, about 77% by weight, about 78% by weight, about 79% by weight, about 80% by weight, about 81% by weight, about 82% by weight, about 83% by weight, about 84% by weight, about 85% by weight, about 86% by weight, about 87% by weight, about 88% by weight, about 89% by weight, about 90% by weight, about 91% by weight, about 92% by weight, about 93% by weight, about 94% by weight, about 95% by weight, about 96% by weight, about 97% by weight, about 98% by weight, or about 99% by weight.

In one embodiment, the weight percent of PEG or mPEG in the PLGA-PEG or PLGA-mPEG di-block copolymers is about 1% to 99%, 2% to 95%, 3% to 90%, 4% to 75%, 5% to 50%, 10% to 47%, 15% to 45%, 20% to 42%, or 25 to 45% by weight.

In one embodiment, the weight percent of PEG or mPEG in the PLGA-PEG or PLGA-mPEG di-block copolymers is about 35% by weight.

In one embodiment, the weight percent of mPEG in the PLGA-mPEG di-block copolymer is about 35% by weight.

The present disclosure also provides any of the therapeutic compositions disclosed herein which comprise a therapeutically effective amount of dactinomycin encapsulated in nanoparticles comprising one or more polymers, where the nanoparticles have an average size of about 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, or 200 nm.

The present disclosure also provides any of the therapeutic compositions disclosed herein which comprise a therapeutically effective amount of dactinomycin encapsulated in nanoparticles comprising one or more polymers, where the nanoparticles have an average size of about 100 nm to about 110 nm, about 105 nm to about 115 nm, about 110 nm to about 120 nm, about 115 nm to about 125 nm, about 120 nm to about 130 nm, about 125 nm to about 135 nm, about 130 nm to about 140 nm, about 135 nm to about 145 nm, about 130 nm to about 140 nm, about 135 nm to about 145 nm, about 140 nm to about 150 nm, about 145 nm to about 155 nm, about 150 nm to about 160 nm, about 155 nm to about 165 nm, about 160 nm to about 170 nm, about 165 nm to about 175 nm, about 170 nm to about 180 nm, about 175 nm to about 185 nm, about 180 nm to about 190 nm, about 185 nm to about 195 nm, or about 190 nm to about 200 nm.

In one embodiment, nanoparticle size may be determined using methods known in the art, for example, using a Malvern Nano-ZS zeta sizer.

The present disclosure also provides any of the therapeutic compositions disclosed herein which comprise a therapeutically effective amount of dactinomycin encapsulated in nanoparticles comprising one or more polymers, where the composition further comprises a surfactant.

In one embodiment, the surfactant comprises about 0.01% to about 10.0% by weight of each nanoparticle. In one embodiment, the surfactant comprises about 0.1% to about 5.0% by weight of each nanoparticle. For example, the surfactant comprises about 0.2% to about 4.0% by weight of each nanoparticle, about 0.5% to about 3.0% by weight of each nanoparticle, about 1.0% to about 2.5% by weight of each nanoparticle.

In one embodiment, the surfactant comprises about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 14.7%, about 4.8%, about 4.9%, or about 5.0% by weight of each nanoparticle.

In one embodiment, the surfactant comprises less than 2.0% by weight of each nanoparticle.

The present disclosure also provides any of the therapeutic compositions disclosed herein which comprise a therapeutically effective amount of dactinomycin encapsulated in nanoparticles comprising one or more polymers, wherein the composition further comprises a surfactant, wherein the surfactant is polyvinyl alcohol, Tween® 20, Tween® 80, vitamin E TPGS, sodium cholate, bile salts (e.g., sodium taurodexoycholate), polyethylene glycol, (e.g., PEG 600, PEG, PEG 4500, Brij® (e.g., 20, 35, 58, and the like), and/or poloxamers (e.g., polaxamer 188, polaxmer 407. In one embodiment, the surfactant is polyvinyl alcohol. In one embodiment, the surfactant is polyvinyl alcohol where the polyvinyl alcohol is 80% hydrolyzed and has a molecular weight of about 9,000 to about 10,000 Da.

The present disclosure also provides any of the therapeutic compositions disclosed herein which comprise a therapeutically effective amount of dactinomycin encapsulated in nanoparticles comprising one or more polymers, wherein the composition further comprises about 1% by weight to about 50% by weight dactinomycin.

The present disclosure also provides any of the therapeutic compositions disclosed herein which comprise a therapeutically effective amount of dactinomycin encapsulated in nanoparticles comprising one or more polymers, wherein the composition further comprises about 1%, 2%, 3%, 4%, 5%, 6%, 7%, 8%, 9%, 10, 1%, 12%, 13%, 14%, 15%, 16%, 17%, 18%, 19%, 20%, 2%1%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight dactinomycin.

The present disclosure also provides any of the therapeutic compositions disclosed herein which comprise a therapeutically effective amount of dactinomycin encapsulated in nanoparticles comprising one or more polymers, wherein the composition further comprises about 5% by weight to about 15% by weight dactinomycin.

The present disclosure also provides therapeutic compositions comprising a therapeutically effective amount of dactinomycin encapsulated in nanoparticles comprising phospholipids, which function as a liquid surfactant which enables the formation of liquid crystals, further comprising purified water, a buffering agent (e.g., glycine), an oil component (e.g., D,L-alpha-tocopherol, and a surfactant (e.g., sodium deoxycholate or polyvinyl alcohol). Examples of phospholipids for these compositions include, without limitation, phosphatidylcholine with negatively charged phospholipids (Phospholipon® 80), lecithin fraction enriched with phosphatidylcholine (Phospholipon® 85G), and phosphatidylcholine stabilized with 0.1% ascorbyl palmitate (Phospholipon® 90G). (See, e.g., U.S. Pat. No. 7,713,440; Particle Sciences Technical Brief, 2012, Vol. 4; and Anderson, D. et al. CRC Concise Encyclopedia of Nanotechnology, Taylor & Francis Group, LLC, 2016, 5 pages, all of which are incorporated herein by reference in their entireties).

In one embodiment, a therapeutic composition comprising a therapeutically effective amount of dactinomycin encapsulated in nanoparticles comprising phospholipids may be prepared by using any methods know in the art. For example, mixing a blank phospholipid composition (e.g., 25 mL of a phosolipid composition comprising Phospholipon® 90G, purified water, glycine, D,L-alpha-tocopherol, sodium decarboyxlate) with dactinomycin (e.g., 375 mg) by vigorous stirring for several hours (e.g., 5 hours), followed by filtration using a 0.45 um CA (cellulose acetate) filter.

The present disclosure provides therapeutic compositions comprising dactinomycin encapsulated in nanoparticles in combination with at least one pharmaceutically acceptable excipient or carrier.

A dactinomycin composition encapsulated in nanoparticles of the disclosure is formulated to be compatible with its intended route of administration. Examples of routes of administration include intravenous administration. Solutions or suspensions used for intravenous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The dactinomycin composition encapsulated in nanoparticles of the disclosure may be manufactured by conventional means, e.g., in a manner as disclosed in McCall, R. L. et al. J. Vis. Exp. (82), e51015.

Any of the dactinomycin compositions encapsulated in nanoparticles disclosed herein can be prepared according to the following general procedure:

A stock solution of dactinomycin (25 mg/mL) is prepared in an organic solvent.

A stock solution of the polymer (e.g. 25 mg/mL when using PLA polymers such as Resomer® R 202S and R 203H, or a 50 mg/mL when using Resomer® D5050 DLG mPEG 5000 (35 wt %, PEG) and 100 DL mPEG 5000 (25 wt %, PEG)) is prepared in an organic solvent.

The dactinomycin solution is added into polymer solution until the amount of the polymer to API reaches 10:1 ratio (w/w).

The final solution is vortexed until homogeneous.

A surfactant solution in water (a water phase) is prepared.

Formation of a dactinomycin nanoparticle emulsion is accomplished by adding the polymer/dactinomycin solution into small amount of water phase while the water phase is on high vortex until the entire polymer solution is added to reach an organic:water phase ratio of about 1:7 by volume.

Continued vortexing followed by transferring the mixture to an ultrasonicator at about 0° C. and sonication for several minutes until desired nanoparticle size is achieved (e.g., about 100 nm to about 200 nm).

Pouring into stirring bulk water phase of surfactant in solution and vigorous stirring at room temperature until complete evaporation of the organic solvent is completed.

The encapsulation efficiency of this procedure is about 1-50%, e.g., 30%, For example, if the procedure is performed using 100 mg of free dactinomycin, about 1-50 mg (e.g., about 30 mg) of the dactinomycin will be encapsulated in nanoparticles.

In one embodiment, any of the dactinomycin compositions encapsulated in nanoparticles disclosed herein can be purified by centrifuging hardened or cured nanoparticles, removing the supernatant, washing the dactinomycin nanoparticles with ddH2O, followed by further centrifuging for several minutes to provide a pellet.

In one embodiment, any of the dactinomycin compositions encapsulated in nanoparticles disclosed herein can be purified to remove free dactinomycin by centrifuging hardened or cured nanoparticles, removing the supernatant, washing the dactinomycin nanoparticles with ddH2O, followed by further centrifuging for several minutes to provide a pellet.

In one embodiment, any of the dactinomycin compositions encapsulated in nanoparticles disclosed herein can be purified to remove 95%, 96%, 97%, 98%, 99%, or more of the free (non-encapsulated) dactinomycin remaining in the composition. Thus, in any of the compositions or formulations described herein, the amount of free dactinomycin is less than about 5% (i.e., less than 5%, less than 4%, less than 3%, less than 2%, less than 1%, less than 0.5%, less than 0.1%) by weight of the amount of dactinomycin encapsulated in nanoparticles. In one embodiment, larger nanoparticle removal from any of the dactinomycin compositions encapsulated in nanoparticles disclosed herein is accomplished by resuspending the pellet in ddH2O and centrifuging for several minutes, followed by collecting the supernatant and then concentrating using a centrifugal filter, and centrifuging for several additional minutes at 14,000× rpm to remove free dactinomycin.

In one embodiment, the dactinomycin compositions encapsulated in nanoparticles prepared in this manner can be used directly.

In one embodiment, any of the dactinomycin compositions encapsulated in nanoparticles disclosed herein may be stored at 4° C. for up to several weeks.

In one embodiment, the dactinomycin compositions encapsulated in nanoparticles disclosed herein may be lyo- and cryo-protected with sucrose (10-30%) prior to storage and lyophilized prior to use. Any other suitable cryo-preservation agent known in the art may also be used.

Drug loading dactinomycin compositions encapsulated in nanoparticles disclosed herein was tested using HPLC.

Further guidance on the preparation of ActD nanoparticle formulation can be found in in McCall, R. L. et al. J. Vis. Exp. (82), e51015, which is incorporated by reference in its entirety.

The dactinomycin compositions encapsulated in nanoparticles of the disclosure may be manufactured on a larger scale. To facilitate this process, a high pressure homogenizer may be used instead of ultrasonification to make the nanoparticles. Further, tangential flow filtration/diafiltration may be used to remove free dactinomycin and concentrate the nanoparticles instead of centrifugation. Also, the addition of lyoprotectant excipients, e.g., sucrose, trehalose, mannitol, and the like may be added to the nanoparticles upon storage, and then lyophilized before use.

Dactinomycin may be capable of forming salts. All of these salt forms are also contemplated within the scope of the disclosure.

Dactinomycin may also be prepared as an ester, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

Dactinomycin may also be prepared as prodrugs, for example, pharmaceutically acceptable prodrugs. The terms “pro-drug” and “prodrug” are used interchangeably herein and refer to any compound which releases an active parent drug in vivo. Prodrugs enhance numerous desirable qualities of pharmaceuticals (e.g., solubility, bioavailability, manufacturing, etc.). Accordingly, the compounds of the disclosure may be delivered in prodrug form. Thus, the disclosure is intended to cover prodrugs of the presently claimed compounds, methods of delivering the same and compositions containing the same. “Prodrugs” are intended to include any covalently bonded carriers that release an active parent drug of the disclosure in vivo when such prodrug is administered to a subject. Prodrugs in the disclosure are prepared by modifying functional groups present in the compound in such a way that the modifications are cleaved, either in routine manipulation or in vivo, to the parent compound. Prodrugs include compounds of the disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like, See Bundegaard, H., Design of Prodrugs, p1-92, Elsevier, New York-Oxford (1985). Therapeutic Administration and Formulation of huIL-6R Antibodies

The antibodies of the disclosure (also referred to herein as “active compounds”), and derivatives, fragments, analogs and homologs thereof, can be incorporated into pharmaceutical compositions suitable for administration. Principles and considerations involved in preparing such compositions, as well as guidance in the choice of components are provided, for example, in Remington's Pharmaceutical Sciences: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Such compositions typically comprise the antibody and a pharmaceutically acceptable carrier. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)).

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated.

The formulations to be used for in vivo administration must be sterile. This is readily accomplished by filtration through sterile filtration membranes.

A composition of the disclosure is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

The active compound is administered by nasal inhalation, inhalation through the mouth, intravenously, orally, or any combination thereof. Alternatively the, active compound is administered orally via an enteric-coated capsule.

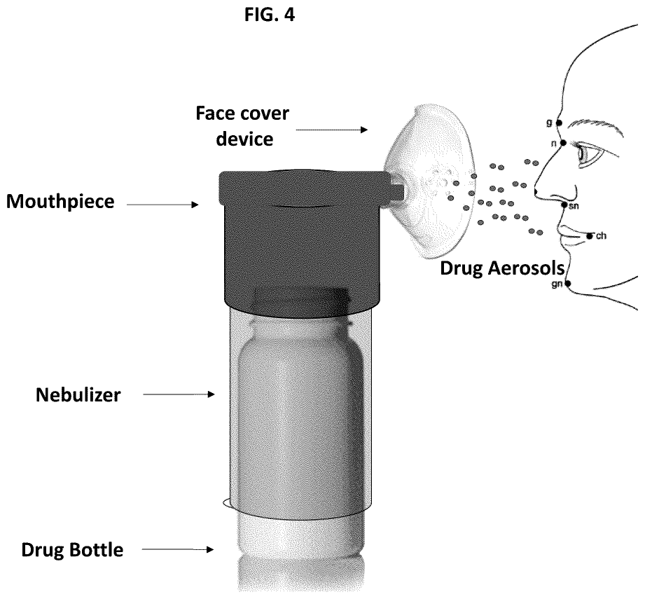

Administration by inhalation may be in the form of an inhaler or a nebulizer (FIG. 4). The nebulizer and/or inhaler is handheld. Optionally, the nebulizer and/or inhaler can be of different sizes to fit children and/or adults.

The active compound may be formulated as a particle and keep the drug particle at a desired range of particle sizes. The particle size range is, for example, between 2-10 microns.

In some embodiments, a formulation, or composition, of the disclosure contains excipients such as stabilizers, preservative, phospholipids and/or other ingredients to improve stability and shelf life and in the case of particles a uniform particle size. In some embodiments, the formulation or composition is a pharmaceutically acceptable composition. Exemplary excipients include, but are not limited to surfactants such as Trehalose (1-20%), emulsifiers such as polysorbate 20 (0.01%-0.1%) or polysorbate 80 (0.01%-0.1%), sodium chloride (50-200 mM), EDTA or EGTA (0.1 to 1 mM), a buffer such as histidine buffer (1-50 mM) or sodium citrate buffer (10-50 mM). Acceptable pH ranges for formulations can be, for example, 4.0 to 7.0.

In some embodiments, the composition comprises an IL-6R antibody, histidine, sodium chloride, and polysorbate 80. In some embodiments, the composition comprises an IL-6R antibody, histidine, sodium chloride, and polysorbate 20. In some embodiments, the histidine is about 5 mM, about 10 mM, about 15 mM, about 20 mM, about 25 mM, about 30 mM, about 35 mM, or about 40 mM. In some embodiments, the histidine is about 25 mM. In some embodiments, the sodium chloride is about 50 mM, about 75 mM, about 100 mM, about 125 mM, about 150 mM, about 175 mM or about 200 mM. In some embodiments, the sodium chloride is about 125 mM. In some embodiments, the polysorbate 80 is about 0.01%, about 0.02%, about 0.03%, about 0.04%, about 0.05%, about 0.06%, about 0.07%, about 0.08%, about 0.09%, or about 0.1%. In some embodiments, the polysorbate 80 is about 0.02%. In some embodiments, the polysorbate 80 is about 0.05%. In some embodiments, the composition has a pH of about 5.0, about 6.0, or about 7.0. In some embodiments, the composition has a pH of about 6.0. In some embodiments, the IL-6R antibody is about 5 mg/mL, about 10 mg/mL, about 15 mg/mL, about 20 mg/mL, about 25 mg/mL, about 30 mg/mL, or about 35 mg/mL. In some embodiments, the IL-6R antibody is about 20 mg/mL. In some embodiments, the histidine is 25 mM, the sodium chloride is 125 mM, the polysorbate 80 is 0.02%, the pH is 6.0, and the IL-6R antibody is 20 mg/mL. In some embodiments, the histidine is 25 mM, the sodium chloride is 125 mM, the polysorbate 80 is 0.05%, the pH is 6.0, and the IL-6R antibody is 20 mg/mL.

In some embodiments, the IL-6R antibody comprises a VH CDR1 region comprising the amino acid sequence of SEQ ID NO: 15, a VH CDR2 region comprising the amino acid sequence of SEQ ID NO: 37, a VH CDR3 region comprising the amino acid sequence of SEQ ID NO: 35, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26.

In some embodiments, the composition comprises IL-6R antibody between about 5 mg/mL and 50 mg/mL. In some embodiments, the composition comprises IL-6R antibody at about 5 mg/mL. In some embodiments, the composition comprises IL-6R antibody at about 10 mg/mL. In some embodiments, the composition comprises IL-6R antibody at about 15 mg/mL. In some embodiments, the composition comprises IL-6R antibody at about 20 mg/mL. In some embodiments, the composition comprises IL-6R antibody at about 25 mg/mL. In some embodiments, the composition comprises IL-6R antibody at about 30 mg/mL. In some embodiments, the composition comprises IL-6R antibody at about 35 mg/mL. In some embodiments, the composition comprises IL-6R antibody at about 40 mg/mL. In some embodiments, the composition comprises IL-6R antibody at about 45 mg/mL. In some embodiments, the composition comprises IL-6R antibody at about 50 mg/mL.

In some embodiments, a vial containing a stabilized and formulated solution or composition of anti-IL-6 receptor (anti-IL-6R) mAb and combinations as described herein is inserted into an inhaler and/or nebulizer. In some embodiments, a vial containing a stabilized and formulated solution of anti-IL-6 receptor (anti-IL-6R) mAb and combinations as described herein is inserted into the bottom of the inhaler and/or nebulizer. In some embodiments, the drug solution is dispensed as fine aerosols through the mouth.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a sustained/controlled release formulations, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

For example, the active ingredients can be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The materials can also be obtained commercially from Alza Corporation and Nova Pharmaceuticals, Inc. Liposomal suspensions (including liposomes targeted to infected cells with monoclonal antibodies to viral antigens) and can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### Combination Therapies

The formulations, compositions, and their associated methods of use can include more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

In one embodiment, the active compounds in a formulation, composition, and their associated methods of use are administered in combination therapy, i.e., combined with other agents, e.g., therapeutic agents, that are useful for coronavirus infection. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

In one embodiment, the active compound is an IL-6R antibody. In one embodiment, the active compounds are IL-6R antibody and anti-CD3 antibody. In one embodiment, the active compounds are IL-6R antibody, anti-CD3, and dactinomycin. In one embodiment, the active compounds are administered in combination with one or more therapeutic agents used in the treatment of coronaviral infection. In one embodiments, the therapeutic agent is bamlanivimab. In one embodiment, the therapeutic agent is etesevimab. In some embodiments, the therapeutic agent is casirivimab. In some embodiments, the therapeutic agent is imdevimab. In some embodiments, the therapeutic agent is remdesivir. In some embodiments, the therapeutic agent in dexamethasone.

For example, the combination therapy can include one or more antibodies of the disclosure coformulated with, and/or coadministered with, one or more additional therapeutic agents, e.g., antiviral drugs, immune booster drugs, Vitamin C or Vitamin D. Such combination therapies may advantageously utilize lower dosages of the administered therapeutic agents, thus avoiding possible toxicities or complications associated with the various monotherapies.

Preferred therapeutic agents used in combination with an antibody of the disclosure are those agents that interfere at different stages in a coronavirus infection. In one embodiment, one or more antibodies described herein may be coformulated with, and/or coadministered with, one or more additional agents.

In some aspects, the anti-IL-6R mAbs used according to any of the embodiments described herein can be, for example, any of the fully human or humanized anti-IL-6R mAbs described herein. In addition, the anti-IL-6R mAb can be Actemra® (tocilizumab) or Kevzera® (sarilumab).

In some embodiments, the anti-IL-6R mAb is the sole antibody in the formulation. Alternatively, the anti-IL-6R mAb is used in combination with one or more of the following: an anti-TNFα antibody (e.g., Humira or Remicade), an anti-CD20 mAb (e.g., Rituximab or Rituxan); an anti-IFNγ mAb (e.g., Gamifant or emapalumab); an anti-Granulocyte-Macrophage Colony-Stimulating Factor mAb (anti-GM-CSF mAb; e.g., GSK3196165) or an anti-CD3 mAbs (e.g., Foralumab).

In various aspects, the anti-IL-6R mAb is administered in combination with an antiviral drug, an immune booster drug, vitamin C, Vitamin D, Vitamin E or any combination thereof.

The anti-viral drug is Remdesivir or Actinomycin D (i.e., dactinomycin).

In some embodiments, a subject is administered anti-IL-6R delivered by inhalation and antiviral drug, such as Actinomycin D, delivered intravenously in a nanoparticle formulation. The combination use of an anti-IL-6R mAb and Actinomycin D produces a synergistic effect that reduces symptoms associated with a coronavirus disease, such as COVID-19, SARS, or MERS.

In some embodiments, a subject is administered anti-IL-6R delivered by inhalation and an anti-CD3 antibody. In some embodiments, the anti-CD3 antibody is delivered intravenously. In some embodiments, the anti-CD3 antibody is delivered by inhalation. In some embodiments, the anti-CD3 is delivered orally. In some embodiments, the anti-CD3 is delivered subcutaneously. The combination use of an anti-IL-6R mAb and anti-CD3 produces a synergistic effect that reduces symptoms associated with a coronavirus disease, such as COVID-19, SARS, or MERS.

In some embodiments, a subject is administered anti-IL-6R delivered by inhalation, and a combination of additional therapeutic agents. In some embodiments, the additional therapeutic agents are dactinomycin and an anti-CD3 antibody. The combination use of an anti-IL-6R mAb, dactinomycin, and anti-CD3 produces a synergistic effect that reduces symptoms associated with a coronavirus disease, such as COVID-19, SARS, or MERS.

### Broad Spectrum Polymeric COVID-19 Antigen

The disclosure further provides a broad spectrum and polymeric peptides antigen derived from the S protein of COVID-19 virus and methods of producing anti-COVID-19 antibodies by administering to a subject broad spectrum and polymeric COVID-19 antigen. The peptides may be conjugated to nanoparticles or suitable carriers with different immunogenic peptides designed from S protein. During the entry of virus, the spike (S) protein of COVID-19 binds to human Angiotensin Converting Enzyme 2 (ACE-2), a putative receptor for COVID-19 (FIG. 4). ACE 2 is a type I transmembrane metallo-carboxypeptidase which is relatively conserved in its protein structure. The S protein of COVID-19 share good homology with S proteins of other RNA viruses belonging to the family Coronaviridae such as SARS-CoV-1, SARS and MERS. Several independent research groups have shown that COVID-19, SARS and MERS utilizes ACE-2 as a cellular entry receptor. Thus, this disclosure provides the use of S protein of COVID-19 for development of an immediate antidote for patients already infected with the virus. Similarly, S protein can also be used as an antigen to produce a vaccine that would potentially protect humans against COVID-19, SARS and MERS.

In some aspects, the S protein derived polymeric antigen may be administered to healthy subjects to produce broad-spectrum polyclonal antibodies against S protein. Such hyperimmune serum, also known as intravenous immunoglobulins (IVIg), from human plasma were first used in 1952 to treat primary immune deficiency. Intravenous immunoglobulin (IVIg) contains the pooled immunoglobulin G (IgG) immunoglobulins from the plasma of approximately a thousand or more blood donors.

IVIgs are sterile, purified IgG products manufactured from pooled human plasma and typically contain more than 95% unmodified IgG, which has intact Fc-dependent effector functions and only trace amounts of immunoglobulin A (IgA) or immunoglobulin M (IgM). Some asymptomatic patients of COVID-19 are carriers of the virus but do not show symptoms of COVID-19 infection because their serum contains antibodies against the virus. Hence, sera of human subjects immunized with S protein of COVID-19 may have neutralizing antibodies against S protein circulating in the blood. Sera from these subjects can be manufactured and purified under sterile conditions and used as an antidote against COVID-19. In other aspects, the method further includes isolating immunoglobulin from the subject, where the immunoglobin binds and neutralized COVID-19.

### Methods of Treating CRS, ARDS and Coronavirus Infections

The disclosure provides methods of treating, preventing, or alleviating a symptom of a CRS, ARDS or a coronavirus infection in a subject in need thereof comprising administering to the subject a composition comprising an IL-6R antibody.

The disclosure further provides methods of treating, preventing, or alleviating a symptom of CRS, ARDS, or a coronavirus infection in a subject in need thereof comprising administering to the subject: a composition comprising an IL-6R antibody and a composition comprising dactinomycin nanoparticles, wherein administration of the composition comprising an IL-6R antibody and administration of the composition comprising dactinomycin nanoparticles can occur in any order or simultaneously.

The disclosure provides methods of treating, preventing, or alleviating a symptom of a pulmonary inflammatory disease in a subject in need thereof comprising administering to the subject a composition comprising an IL-6R antibody.

The disclosure further provides methods of treating, preventing, or alleviating a symptom of a pulmonary inflammatory disease in a subject in need thereof comprising administering to the subject: a composition comprising an IL-6R antibody and a composition comprising dactinomycin nanoparticles, wherein administration of the composition comprising an IL-6R antibody and administration of the composition comprising dactinomycin nanoparticles can occur in any order or simultaneously.

The IL-6R antibodies described herein may be used as therapeutic agents. Such agents will generally be employed to treat, alleviate, and/or prevent a disease or pathology associated with coronavirus infection in a subject. A therapeutic regimen is carried out by identifying a subject, e.g., a human patient suffering from (or at risk of developing) coronavirus infection, using standard methods. In some embodiments, the subject has a disease or pathology associated with coronavirus infection. In some embodiments, the subject has a disease of pathology associated with MERS and/or its variants. In some embodiments, the subject has a disease or pathology associated with SARS and/or its variants. In some embodiments, the subject has a disease or pathology associated with a pulmonary inflammatory disease. Examples of pulmonary inflammatory diseases include ARDS (acute respiratory distress syndrome) and systemic pulmonary sclerosis.

An IL-6R antibody preparation, preferably one having high specificity and high affinity for its target antigen, is administered to the subject and will generally have an effect due to its binding with the target. Administration of the antibody abrogates or inhibits or interferes with the signaling function of the target (e.g., IL-6Rc).

Administration of the antibody abrogates or inhibits or interferes with the binding of the target (e.g., IL-6Rc) with an endogenous ligand (e.g., gp130) to which it naturally binds. For example, the antibody binds to the target and modulates, blocks, inhibits, reduces, antagonizes, neutralizes, or otherwise interferes with IL-6 signaling. In some embodiments, the IL-6 antibodies are administered by inhalation. In some embodiments, the IL-6R antibodies are administered intravenously. In some embodiments, the IL-6R antibodies are delivered orally. In some embodiments, the IL-6R antibodies are delivered subcutaneously.

In some embodiments, the methods of treatment provided herein include co-administering the IL-6R antibodies with another therapeutic agent. In some embodiments, the IL-6R antibodies are co-administered with dactinomycin nanoparticles. In some embodiments, the IL-6R antibodies of the disclosure are administered before, simultaneously, or following administration of dactinomycin nanoparticles. When administered simultaneously the IL-6R antibodies and the dactinomycin nanoparticles can be formulated together or separately and administered as described herein In some embodiments, the methods of treatment provided herein include co-administering the IL-6R antibodies with a monoclonal antibody. In some embodiments, the monoclonal antibody is an anti-CD3 antibody. In some embodiments, the methods of treatment provided herein include co-administering anti-IL-6R delivered by inhalation and anti-CD3 antibodies. In some embodiments, the anti-CD3 antibodies are delivered intravenously. In some embodiments, the anti-CD3 antibodies are delivered by inhalation. In some embodiments, the anti-CD3 antibodies are delivered orally. In some embodiments, the anti-CD3 antibodies are delivered subcutaneously.

In some embodiments, the anti-CD3 antibody is foralumab. In some embodiments, the anti-CD3 antibody comprises comprise a heavy chain complementarity determining region 1 (CDRH1) comprising the amino acid sequence (SEQ ID NO: 42), a heavy chain complementarity determining region 2 (CDRH2) comprising the amino acid sequence (SEQ ID NO: 43), a heavy chain complementarity determining region 3 (CDRH3) comprising the amino acid sequence (SEQ ID NO: 44), a light chain complementarity determining region 1 (CDRL1) comprising the amino acid sequence (SEQ ID NO: 45), a light chain complementarity determining region 2 (CDRL2) comprising the amino acid sequence (SEQ ID NO: 46), and a light chain complementarity determining region 3 (CDRL3) comprising the amino acid sequence (SEQ ID NO: 47). In some embodiments, the anti-CD3 antibody comprises a variable heavy chain amino acid sequence comprising (SEQ ID NO: 48) and a variable light chain amino acid sequence comprising (SEQ ID NO: 49). In some embodiments, the anti-CD3 antibody comprises a heavy chain amino acid sequence comprising: (SEQ ID NO: 50) and a light chain amino acid sequence comprising: (SEQ ID NO: 51). The combination use of an anti-IL-6R mAb and anti-CD3 produces a synergistic effect that reduces symptoms associated with a coronavirus disease, such as COVID-19, SARS, or MERS.

In some embodiments, the method of treatment comprises co-administering anti-IL-6R antibodies and a combination of additional therapeutic agents. In some embodiments, the additional therapeutic agents are dactinomycin and an anti-CD3 antibody. The combination use of an anti-IL-6R mAb, dactinomycin, and anti-CD3 produces a synergistic effect that reduces symptoms associated with a coronavirus disease, such as COVID-19, SARS, or MERS.

In some embodiments, the subject is a human subject. In some embodiments, the subject has or is suspected of having a coronavirus infection. In some embodiments, the subject has been or thought to have been exposed to a coronavirus and has not yet developed symptoms of a coronavirus infection. In some embodiments, the coronavirus is, for example, the virus that causes COVID-19, SARS, or MERS. Signs and symptoms of a coronavirus infection can be, for example, fever, cough, and shortness of breath.

The active compound is administered by nasal inhalation, inhalation through the mouth, intravenously, orally, any combination thereof or any other route of administration described herein. Alternatively the, active compound is administered orally via an enteric-coated capsule.

Administration by inhalation may be in the form of an inhaler or a nebulizer. The nebulizer and/or inhaler is handheld. Optionally, the nebulizer and/or inhaler can be of different sizes to fit children and/or adults.

In therapeutic applications, the dosages of the dactinomycin nanoparticle compositions used in accordance with the disclosure vary depending on the age, weight, clinical condition of the recipient patient, the severity of the condition to be treated; the route of administration; the renal and hepatic function of the patient; the particular compound or salt thereof employed. An ordinarily skilled physician or veterinarian can readily determine and prescribe the effective amount of the active compound required to prevent, counter or arrest the progress of the condition.

Generally, the dose should be sufficient to result in slowing the replication of the coronavirus within the host and also preferably prevent or reduce the symptoms of a coronavirus-related disease (e.g. COVID-19, SARS, or MERS). The dose chosen should be sufficient to constitute effective treatment but not as high as to cause unacceptable side effects (e.g., mucositis or anaphylactic shock). The state of the disease condition (e.g., SERS, MERS, or COVID-19) and the health of the patient should preferably be closely monitored during and for a reasonable period after treatment.

Dosages of IL-6R antibodies administered by nebulizer or inhaler can range from about 10 mg to about 100 mg. In some embodiments, the dose is about 20 mg to about 100 mg. In some embodiments, the dose is about 30 mg to about 100 mg. In some embodiments, the dose is about 40 mg to about 100 mg. In some embodiments, the dose is about 50 mg to about 100 mg. In some embodiments, the dose is about 60 mg to about 100 mg. In some embodiments, the dose is about 70 mg to about 100 mg. In some embodiments, the dose is about 80 mg to about 100 mg. In some embodiments, the dose is about 90 mg to about 100 mg. In some embodiments, the dose is about 25 to about 75 mg. In some embodiments, the dose is about 25 mg. In some embodiments, the dose is about 50 mg. In some embodiments, the dose is about 75 mg.

In some embodiments, the IL-6R antibody administered by nebulizer or inhaler comprises In some embodiments, the IL-6R antibody comprises a VH CDR1 region comprising the amino acid sequence of SEQ ID NO: 15, a VH CDR2 region comprising the amino acid sequence of SEQ ID NO: 37, a VH CDR3 region comprising the amino acid sequence of SEQ ID NO: 35, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26.

Dosages of IL-6R antibodies can range from about 1 μg/kg per day to about 30 μg/kg per day in a single, a divided, or a continuous dose (which dose may be adjusted for the patient's weight in kg, body surface area in m², and age in years).

In one embodiment, the compositions and methods of the disclosure include those where the therapeutically effective amount of dactinomycin nanoparticles results in an AUC∞ of about 0.05 to about 2.0 mg*min/L.

In one embodiment, the compositions and methods of the disclosure include those where the therapeutically effective amount of dactinomycin nanoparticles results in an AUC∞ of about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.05, 1.10, 1.15, 1.20, 10.25, 1.30, 1.35, 1.40, 1.45, 1.50, 1.55, 1.60, 1.65, 1.70, 1.75, 1.80, 1.85, 1.90, 1.95, or 2.0 mg*min/L.

In one embodiment, the compositions and methods of the disclosure include those where the therapeutically effective amount of dactinomycin nanoparticles results in an AUC∞ of about 0.05 to about 1.0 mg*min/L.

In one embodiment, the compositions and methods of the disclosure include those where the therapeutically effective amount of dactinomycin nanoparticles results in a Cmax of about 1 ng/mL to about 30 ng/mL.

In one embodiment, the compositions and methods of the disclosure include those where the therapeutically effective amount of dactinomycin nanoparticles results in a Cmax of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30 ng/mL.

In one embodiment, the compositions and methods of the disclosure include those where the therapeutically effective amount of dactinomycin nanoparticles results in a Cmax of about 1 ng/mL to about 20 ng/mL.

In one embodiment, the compositions and methods of the disclosure include those where the therapeutically effective amount of dactinomycin nanoparticles results in a Cmax of about 1 ng/mL to about 5 ng/mL, about 5 ng/mL to about 10 ng/mL, about 10 ng/mL to about 15 ng/mL, or about 15 ng/mL to about 20 ng/mL.

In one embodiment, the compositions and methods of the disclosure include those where about 60% to about 90% of the dactinomycin is released from the nanoparticles after 2, 3, 4 or 5 days.

In one embodiment, the compositions and methods of the disclosure include those where about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the dactinomycin is released from the nanoparticles after 2, 3, 4, or 5 days.

In one embodiment, the compositions and methods of the disclosure include those where about 60% to about 90% of the dactinomycin is released from the nanoparticles after 3 days.

In one embodiment, the compositions and methods of the disclosure include those where about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the dactinomycin is released from the nanoparticles after 3 days.

In one embodiment, the compositions and methods of the disclosure include those where about 70% to about 80% of the dactinomycin is released from the nanoparticles after 2, 3, 4, or 5 days.

In one embodiment, the compositions and methods of the disclosure include those where about 70% to about 80% of the dactinomycin is released from the nanoparticles after 3 days.

Dactinomycin nanoparticles may be administered between 1 and 30 μg/kg/day, inclusive of the endpoints. In certain embodiments, compositions of the disclosure comprising dactinomycin may be administered between 10 and 20 μg/kg/day or between 10 and 15 μg/kg/day, inclusive of the endpoints.

Dactinomycin nanoparticles may be administered at about 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day, 10 μg/kg/day, 11 μg/kg/day, 12 μg/kg/day, 13 μg/kg/day, 14 μg/kg/day, 15 μg/kg/day, 16 μg/kg/day, 17 μg/kg/day, 18 μg/kg/day, 19 μg/kg/day, 20 μg/kg/day, 21 μg/kg/day, 22 μg/kg/day, 23 μg/kg/day, 24 μg/kg/day, 25 μg/kg/day, 26 μg/kg/day, 27 μg/kg/day, 28 μg/kg/day, 29 μg/kg/day, or 30 μg/kg/day.

Dactinomycin nanoparticles may be administered at about 5 μg/kg/day to about 15 μg/kg/day. In certain embodiments dactinomycin nanoparticles may be administered in a therapeutically effective amount of the composition of about 12.5 μg/kg/day. In certain embodiments, dactinomycin nanoparticles may be administered at about 12.5 μg/kg/day.

An effective amount of a pharmaceutical agent is that which provides an objectively identifiable improvement as noted by the clinician or other qualified observer. For example, slowing the replication of the coronavirus may be detected using PCR amplification assay to detect coronavirus nucleotides. Efficacy is also indicated by reduction in symptoms such as dry cough, pneumonia-like symptoms, or fever,

Long-acting compositions may be administered every 3 to 4 days, every week, or once every two weeks depending on half-life and clearance rate of the particular formulation.

The dosage regimen utilizing the therapeutic compounds is selected in accordance with a variety of factors including type, species, age, weight, sex and medical condition of the patient; The dosage regimen can be daily administration (e.g. every 24 hours) of a therapeutic compound of the disclosure. The dosage regimen can be daily administration for consecutive days, for example, at least two, at least three, at least four, at least five, at least six or at least seven consecutive days. Dosing can be more than one time daily, for example, twice, three times or four times daily (per a 24 hour period). The dosing regimen can be a daily administration followed by at least one day, at least two days, at least three days, at least four days, at least five days, or at least six days, without administration. For example, a therapeutic compound of the disclosure is administered at least once in a 24 hour period, then a compound of the disclosure is not administered for at least six days, then a therapeutic compound of the disclosure is administered to a subject in need.

The dosing regimen can be once or twice over a period of 1, 2, or 3 weeks.

In one embodiment, a representative dosing cycle comprises administration of an IL-6R antibody followed by treatment with any of the nanoparticle compositions of the application once or twice over a period of days or 1, 2, or 3 weeks, followed by a drug holiday of one week, two weeks, three weeks, four weeks, five weeks, or six weeks, followed by treatment with any of the nanoparticle compositions of the application once or twice over a period of 1, 2, or 3 weeks.

In some embodiments, the IL-6R antibody dosage is administered in aerosolized form. In some embodiments the IL-6R antibody dosage is administered using a nebulizer or inhaler. In some embodiments, the administration using a nebulizer is every other day for two weeks. In some embodiments, the administration using a nebulizer is single dose. In some embodiments, the administration using a nebulizer is two times in one week. In some embodiments, the administration using a nebulizer is three times in one week. In some embodiments, the administration using a nebulizer is four times in one week. In some embodiments, the administration using a nebulizer is four times in 9 days. In some embodiments, the administration using a nebulizer is five times in 10 days. In some embodiments, the administration using a nebulizer is six times in 12 days. In some embodiments, the administration using a nebulizer is six times in 14 days.

Compositions dactinomycin nanoparticles a composition may be administered once a day or twice a day.

Efficaciousness of treatment is determined in association with any known method for diagnosing or treating coronavirus infection. Alleviation of one or more symptoms of coronavirus infection indicates that the antibody confers a clinical benefit.

The compositions can be included in a container, pack, or dispenser together with instructions for administration.

In one embodiment, antibodies of the disclosure, which include a monoclonal antibodies (e.g., a fully human monoclonal antibody), may be used as therapeutic agents. Such agents will generally be employed to treat, alleviate, and/or prevent a disease or pathology associated with coronavirus infection in a subject. A therapeutic regimen is carried out by identifying a subject, e.g., a human patient suffering from (or at risk of developing) coronavirus infection, using standard methods. An antibody preparation, preferably one having high specificity and high affinity for its target antigen, is administered to the subject and will generally have an effect due to its binding with the target. Administration of the antibody abrogates or inhibits or interferes with the signaling function of the target (e.g., IL-6Rc). Administration of the antibody abrogates or inhibits or interferes with the binding of the target (e.g., IL-6Rc) with an endogenous ligand (e.g., gp130) to which it naturally binds. For example, the antibody binds to the target and modulates, blocks, inhibits, reduces, antagonizes, neutralizes, or otherwise interferes with IL-6 signaling.

In some embodiments, the subject is a human subject. In some embodiments, the subject has or is suspected of having a coronavirus infection. In some embodiments, the subject has been or thought to have been exposed to a coronavirus and has not yet developed symptoms of a coronavirus infection. In some embodiments, the coronavirus is, for example, the virus that causes COVID-19, SARS, or MERS. Signs and symptoms of a coronavirus infection can be, for example, fever, cough, and shortness of breath.

A therapeutically effective amount of an antibody of the disclosure relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragments described herein may be, by way of nonlimiting example, from about 0.1 mg/kg body weight to about 50 mg/kg body weight. Common dosing frequencies may range, for example, from twice daily to once a week.

Efficaciousness of treatment is determined in association with any known method for diagnosing or treating coronavirus infection. Alleviation of one or more symptoms of coronavirus infection indicates that the antibody confers a clinical benefit.

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used in this specification and the appended claims, the singular forms “a,” “an” and “the” include plural references unless the content clearly dictates otherwise.

As used in this specification, the term “and/or” is used in this disclosure to mean either “and” or “or” unless indicated otherwise.

Throughout this specification, unless the context requires otherwise, the words “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%1, 7%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As utilized in accordance with the present disclosure, the following terms, unless otherwise indicated, shall be understood to have the following meanings:

As used herein, the terms Interleukin-6 Receptor, IL-6R, Interleukin-6 Receptor-alpha, IL-6Ra, cluster differentiation factor 126, and CD126 are synonymous and may be used interchangeably. Each of these terms refers to the homodimeric protein, except where otherwise indicated.

As used herein, the term CD3 refers to any protein in the cluster of differentiation 3 protein complex, including the CD37 chain, a CD36 chain, and/or the CD3F chain. In some instances, CD3 may refer to the CD3 complex, an individual CD3 chain, or a specific epitope on any one of the CD3 chains or on the CD3 complex.

As used herein, the term TNF refers to a tumor necrosis factor protein, which may also be referred to as tumor necrosis factor alpha, TNF alpha, TNFα, and/or TNF-α, each of which may be used interchangeably.

As used herein, the term “coronavirus infection” is refers to infection by enveloped non-demented positive strand RNA virus belonging to the family Coronavirdae. For example, the coronavirus is SARS-CoV, SARS-CoV-2, MERS-CoV, or a mutant and/or variant thereof.

Variants of SARS-CoV-2 can include, without limitation, B.1.351, B.1.1.7, and P.1. It is to be understood that new variants of coronavirus with novel mutations or sets of mutations can arise, and these are also covered by the term “coronavirus infection.”

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically binds” or “immunoreacts with” or “directed against” is meant that the antibody reacts with one or more antigenic determinants of the desired antigen and does not react with other polypeptides or binds at much lower affinity (Kd>10-6). Antibodies include, but are not limited to, polyclonal, monoclonal, chimeric, dAb (domain antibody), single chain, Fab, Fab′ and F(ab′)2 fragments, scFvs, and an Fab expression library.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. mAbs contain an antigen binding site capable of immunoreacting with a particular epitope of the antigen characterized by a unique binding affinity for it.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin or fragment thereof, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant (Kd) is ≤1 M; e.g., ≤100 nM, preferably ≤10 nM and more preferably ≤1 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kon) and the “off rate constant” (Koff) can be determined by calculation of the concentrations and the actual rates of association and dissociation. (See Nature 361:186-87 (1993)). The ratio of Koff/Kon enables the cancellation of all parameters not related to affinity, and is equal to the dissociation constant Kd. (See, generally, Davies et al. (1990) Annual Rev Biochem 59:439-473). An antibody of the present disclosure is said to specifically bind to IL-6Rc and/or both IL-6Rc and IL-6R, when the equilibrium binding constant (K d) is ≤1 M, preferably ≤100 nM, more preferably ≤10 nM, and most preferably ≤100 pM to about ≤1 pM, as measured by assays such as radioligand binding assays or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the disclosure include the nucleic acid molecules encoding the heavy chain immunoglobulin molecules presented in SEQ ID NOS: 2, 8 and 12, and nucleic acid molecules encoding the light chain immunoglobulin molecules represented in SEQ ID NOS: 4, 6, 10, and 14.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the disclosure comprise the heavy chain immunoglobulin molecules represented in SEQ ID NOS: 2, 8, and 12, and the light chain immunoglobulin molecules represented in SEQ ID NOS: 4, 6, 10, and 14 as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g., for probes, although oligonucleotides may be double stranded, e.g., for use in the construction of a gene mutant. Oligonucleotides of the disclosure are either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes Oligonucleotides linkages such as phosphorothioate, phosphorodithioate, phosphoroselerloate, phosphorodiselenoate, phosphoroanilothioate, phoshoraniladate, phosphoronmidate, and the like. See e.g., LaPlanche et al. Nucl. Acids Res. 14:9081 (1986); Stec et al. J. Am. Chem. Soc. 106:6077 (1984), Stein et al. Nucl. Acids Res. 16:3209 (1988), Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). An oligonucleotide can include a label for detection, if desired.

The term “selectively hybridize” referred to herein means to detectably and specifically bind. Polynucleotides, oligonucleotides and fragments thereof in accordance with the disclosure selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments described herein and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland7 Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present disclosure. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and which are 5′ to the 5′ end of the RNA transcript are referred to as “upstream sequences”, sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present disclosure, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the disclosure.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et at. Nature 354:105 (1991).

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full length cDNA sequence. Fragments typically are at least 5, 6, 8 or 10 amino acids long, preferably at least 14 amino acids long’ more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has specific binding to CD3, IL-6Rc and/or both IL-6Rc and IL-6R, under suitable binding conditions. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986), Veber and Freidinger TINS p. 392 (1985); and Evans et al. J. Med. Chem. 30:1229 (1987). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH2NH—, —CH═CH-(cis and trans), —COCH 2-, CH(OH)CH 2-, and —CH 2SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90T, 99Tc, 111In, 1251, 131I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The amino acids encompassing the complementarity determining regions (CDR) are as defined by E. A. Kabat et al. (See Kabat, E A, et al., Sequences of Protein of immunological interest, Fifth Edition, US Department of Health and Human Services, US Government Printing Office (1991)).

The term “anti-inflammatory agent”, as used herein, includes, without limitation, pentoxifylline and nonsteroidal anti-inflammatory drugs (NSAIDs), e.g., ibuprofen.

The term “antioxidant”, as used herein, includes, without limitation, vitamin C, retinoic acid (e.g., all trans-retinoic acid), amifostine, and N-acetyl cysteine.

As used herein, the term “dosage effective manner” refers to amount of an active compound to produce the desired biological effect in a subject.

The term “drug holiday”, as used herein, refers to an amount of time in between administrations of any of the therapeutic composition of the disclosure for the treatment of a coronavirus related disease. In one aspect, the drug holiday is 1 week, 2 weeks, 3 weeks, 4 weeks, or any amount of time in between.

As used herein, the terms “encapsulation,” “encapsulated,” and the like mean that the dactinomycin and/or other drug or agent are encapsulated within the nanoparticles (e.g., fully or partially), associated with the nanoparticles, and/or adsorped on or onto the nanoparticles.

The term “growth and differentiation promoters”, includes, without limitation, glutamine.

A “nanoparticle composition”, “nanoparticle dactinomycin composition” or “dacinomycin composition” is any formulation containing a dactinomycin nanoparticle composition as described herein in a form suitable for administration to a subject. In one embodiment, the composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. Although compositions of the disclosure may be administered by any route, preferred routes of administration include intravenous injection or infusion. In one embodiment, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference.

As used herein, “pharmaceutically acceptable salts” refer to derivatives of the compounds of the disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, meglumine, and the like.

It should be understood that all references to pharmaceutically acceptable salts include solvent addition forms (solvates) or crystal forms (polymorphs) as defined herein, of the same salt.

The term “therapeutically effective amount”, as used herein, refers to an amount of a therapeutic composition to treat, ameliorate, or prevent an identified disease or condition, or to exhibit a detectable therapeutic or inhibitory effect. The effect can be detected by any assay method known in the art. A therapeutically effective amount of an antibody of the disclosure relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the disclosure may be, by way of nonlimiting example, from about 0.1 mg/kg body weight to about 50 mg/kg body weight. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In one aspect, the disease or condition to be treated is associated with coronavirus infection. In one aspect, the disease or condition to be treated is a suspected or diagnosed coronavirus-related disease, such as COVID-19, SERS, or MERS.

For any composition of the disclosure, the therapeutically effective amount can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans.

Therapeutic/prophylactic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Compositions that exhibit large therapeutic indices are preferred. The dosage may vary within this range depending upon the dosage form employed, sensitivity of the patient, and the route of administration.

The term “cytokine” refers to all human cytokines known within the art that bind extracellular receptors expressed on the cell surface and thereby modulate cell function, including but not limited to IL-2, IFN-gamma, TNF-α, IL-4, IL-5, IL-6, IL-9, IL-10, and IL-13.

### EXAMPLES

### Example 1: Formulation of IL-6 Targeting Antibodies for Administration by Aerosolization

A formulation was developed for delivering aerosolized IL-6 targeting antibodies, useful for administration using a nebulizer. Formulations tested included histidine (His), sodium chloride (NaCl), polysorbate 80 (PS80), and the IL-6 targeting antibody at pH 6.0. Two specific formulations were Formulation F4 (20 mg/mL anti-IL-6 antibody, 25 mM His, 125 mM NaCl, 0.02% PS80, pH 6.0) and Formulation F5 (20 mg/mL anti-IL-6 antibody, 25 mM His, 125 mM NaCl, 0.05% PS80, pH 6.0). A control buffer was used consisting of 26 mg/mL IL-6R antibody, 25 mM His, 125 mM NaCl, pH 6.0.

The formulations were frozen on dry ice. The sample formulations were thawed, and 1.5 mL of each was loaded in a nebulizer (Aerogen) and aerosolized. Aerosolized material was collected into 1 mL corresponding buffer. A summary is shown in Table 5.

TABLE 5
Summary of Formulation Volumes in Aerosolization Experiments

| | | Volume | Total Volume | | |
|---|---|---|---|---|---|
| | Volume of | Buffer for | of Nebulized | Volume of | |
| | Formulation | Collecting | Material and | Recovered | |
| Formu- | Loaded Into | Nebulized | Collection | Formu- | % Dilu- |
| lation | device | Material | Buffer | lation | tion |
| F4 | 1.5 | 1 | 2 | 1 | 50% |
| F5 | 1.5 | 1 | 2.3 | 1.3 | 56% |

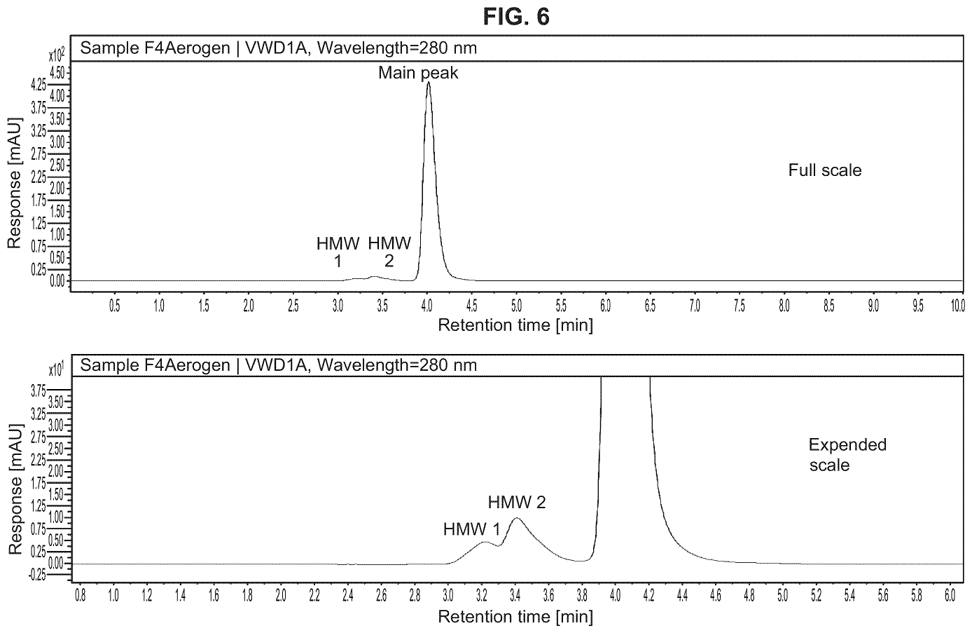

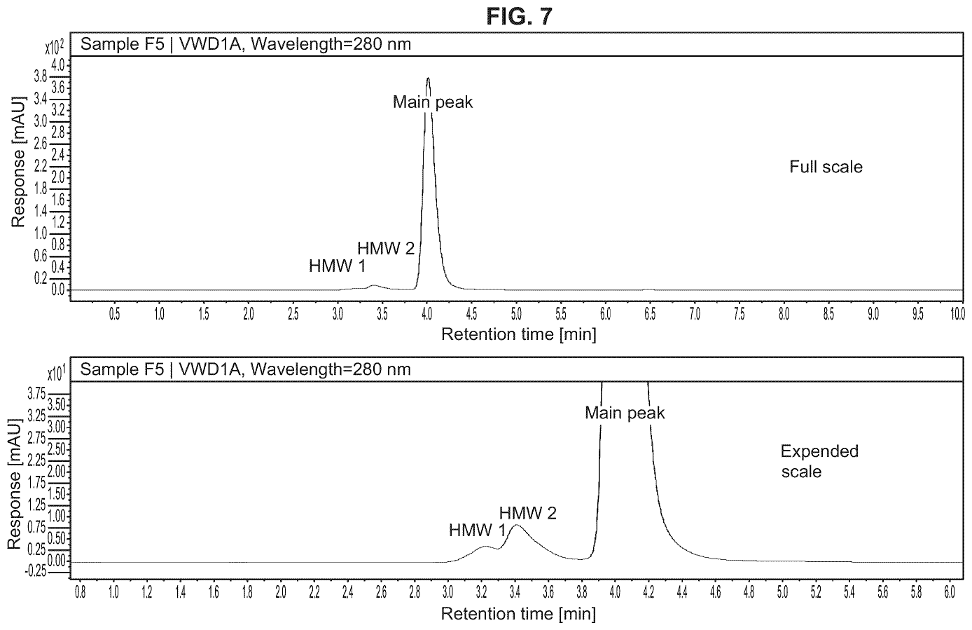

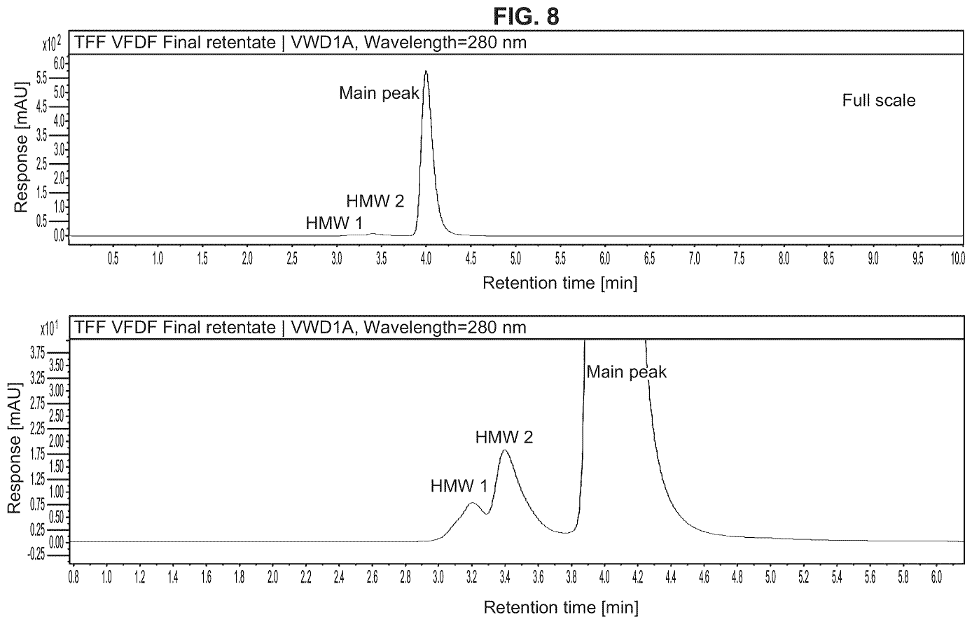

To determine the effect of the polysorbate 80 concentration on IL-6 antibody aggregation in nebulized material, the collected sample was analyzed using size exclusion chromatography. To do this, 2 μL of the collected sample was loaded onto a UPLC SEC column and eluted in elution buffer (0.1M sodium phosphate, 0.2M arginine HCl, pH 7.0) for a 10 minute run time. Chromatographs were generated for each the F4 (FIG. 6), F5 (FIG. 7), and control formulations (FIG. 8). The chromatographs were used to determine a quantitative profile of high molecular weight (HMW) aggregates in the collected samples. A summary of the HMW aggregate profiles for each formulation are shown in Table 6.

TABLE 6
Summary of Aggregation Results in Aerosolization Experiments

| | Area | % | Area | % HMW | Area | | Expected | Relative | Expected |
|---|---|---|---|---|---|---|---|---|---|
| Samples | HMW 1 | HMW | HMW 2 | 2 | Main | % Main | Load (μg) | Conc. | Recovery |
| Control | 80.6 | 0.71 | 226.5 | 1.99 | 11079 | 97.3 | — | — | |
| F4 | 51.1 | 1.2 | 136.2 | 3.1 | 4201 | 95.7 | 38% | 50.1% | 50% |
| F5 | 35.6 | 0.9 | 110.4 | 2.9 | 3691 | 96.2 | 33% | 43.8% | 56% |

The total percent HMW aggregates for control, F4, and F5 formulations were 2.7%, 4.3%, and 3.8%, respectively. In comparison to the control sample, aggregate level increased by 1.6% for F4 and 1.1% for F5, suggesting that increased PS80 concentration in the formulation better protects the anti-IL-6 antibodies during aerosolization.

A formulation containing 0.02% polysorbate 20 (PS20) was developed for comparison, and showed aggregate level increased by 0.7%.

In summary, the results show that levels of polysorbate 80 are important to prevent aggregation (grossly observed at the macro level and by SEC HPLC) resulting from shear at the surface of the mesh nebulizer. Formulation containing PS20 and PS80 were evaluated, and the 0.05% level for PS80 was adequate for maintaining IL-6 antibody stability during aerosolization.

### Example 2: Administration of Aerosolized IL-6R Antibody

The goal of the pulmonary delivery of IL-6R antibody is to deposit safe, tolerable and efficacious doses of IL-6R antibody to the affected parts of the respiratory tract in subjects with SARS-COV2 respiratory tract infections and the COVID-19 sequelae.

An illustrative formulation intended for the use in human clinical trials and ultimately in the commercial product is a buffered aqueous solution of IL-6R antibody stabilized with a small amount of surfactant (25 mM Histidine, 125 mM NaCl, 0.05% Polysorbate 80, pH 6.0). This composition was selected for several reasons. One reason is to maximize the potential for safety and tolerability of the ingredients including the need for near-neutral pH and close to iso-osmolar composition. Another reason is to minimize potential damage to the integrity of IL-6R antibody during the manufacture, storage and use of the drug product.

The delivery system to administer aerosolized IL-6R antibody formulation was selected based on several criteria. Selection criteria include ease of use in patients with COVID-19 infections, particle size distribution to facilitate adequate deposition in the affected parts of the respiratory tract, minimum impact on the integrity of the IL-6R antibody, minimum waste of the IL-6R antibody in the delivery system.

For ease of use by the target patient population, nebulizer delivery was selected. Nebulizer administration of liquid dosage forms does not require any extra breathing effort by the patients as they use simple tidal breathing to inhale the aerosol that is generated by external energy input. Little coordination is required and even ventilated patients can use this type of delivery.

Vibrating mesh nebulizers have been developed as an improvement over jet nebulizers as they deliver the medication faster and more efficiently. The Aerogen Solo vibrating mesh nebulizer was selected because of its substantially higher efficiency of delivery (small amount of residue of the drug post-nebulization). Aerogen Solo is an FDA approved device (510(K) reference).

Several formulations were screened to investigate the impact of aerosolization on the integrity of IL-6R antibody. Protein aggregation in particular is a critical parameter because of its potential adverse immunogenic properties. The prototype formulations (20 mg/mL IL-6R antibody with various buffer components and stabilizers) were tested by comparison of the state of aggregation in the unnebulized formulation vs. formulation collected in the corresponding vehicle buffer after nebulization of 1.5 mL. Using size exclusion chromatography for the analyses, the smallest increase in aggregation of IL-6R antibody was found with the formulation containing 0.05% polysorbate 80: the % of the high molecular weight aggregates increased from 2.7% to 3.8% (see Example 2). This resulted in the selection of the final formulation (25 mM Histidine, 125 mM NaCl, 0.05% PS80, pH 6.0).

To estimate delivered dose of IL-6R antibody, the Aerogen Solo nebulizer was tested in triplicate runs with the selected final formulation containing 25 mg/mL IL-6R antibody. The total delivered dose to the mouthpiece using a unidirectional flow rate of 15 L/min achieved with a vacuum pump connected downstream of in-series connected two collection filters was determined. In each run, three consecutive 1 mL loads of the formulation were individually delivered and collected on filters (the use of three aliquots of volumes of 1 mL within a run was done to prevent overloading of the filters). The nebulizer was then cleaned and dried before another triplicate run.

On average, the delivered dose per 1 mL loaded (i.e., 25 mg) was 23.7 mg IL-6R antibody (94.8% of the loaded dose, range 88.8-100.3%, n=9). No systematic trends within the 3 runs or between the three consecutive runs were observed. It can be therefore concluded that the estimated delivered dose per 1 mL of formulation is 23.7 mg.

For the purpose of estimating the dose delivered to the mouth of patients, it is customary to assume that approximately 50% of the delivered dose would be lost while the subject is exhaling (O'Callaghan and Barry, 1997). Therefore the inhaled dose is estimated to be 11.8 mg/mL per 1 mL of the 25 mg/mL IL-6R antibody formulation.

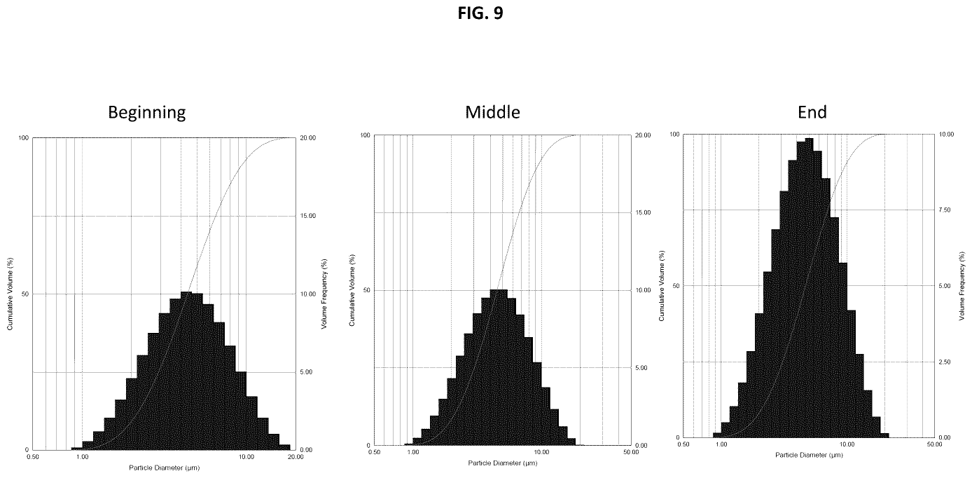

To determine droplet size distribution of the delivered dose, laser diffraction method was used for the droplet size distribution of the aerosol emitted from the Aerogen Solo vibrating mesh nebulizer. Three nebulizers were tested in triplicate with loaded doses of 1 mL of the 25 mg/mL formulation of IL-6R antibody; for each run, the droplet size distribution was measured at the beginning, in the middle and at the end of the nebulization of 1 mL loaded. An illustrative example of the data collected is shown in FIG. 9.

Assuming the density of the formulation to be approximately the same as that of water (i.e., 1 μg/mL), the mass median aerodynamic diameter (MMAD) and the geometric standard deviation (GSD) of the droplet size distributions were calculated. The mass median aerodynamic diameters (MMAD) for the 3 nebulizers from the 3 sampling times (beginning (A), middle (B), and end (C)) were: A: 4.4 μm, 4.5 μm, and 4.7 μm; B: 3.9 μm, 4.4 μm, and 4.7 μm; and C: 4.6 μm, 4.8 μm, and 4.8 μm, with a total average MMAD 4.5 μm. The Geometric Standard Deviations (GSD) for the 3 nebulizers in all runs were all 1.7 μm. The average fine particle fractions below 3.5 μm (FPF<3.5) for the three nebulizers was 33.7% (overall range from all runs 30.3-41.7%).

The lung dose of IL-6R antibody in humans using the in vitro characterization of delivery of the 25 mg/mL formulation with the Aerogen Solo vibrating mesh nebulizer was estimated. The lung dose (LD) in humans is estimated as the product of the inhaled dose multiplied by the Fine Particle Fraction (FPF). Using the in vitro results for inhaled dose and FPF, the lung dose in humans per 1 mL of the IL-6R antibody formulation loaded in the Aerogen Solo vibrating mesh nebulizer is estimated to be 4 mg, i.e., approximately 16% of the loaded dose of 25 mg.

### Example 3: Repeated-Dose Inhalation Toxicity Study Followed by a 14-Day Recovery Period in Cynomolgus Monkeys

### Study Objective and Experimental Design

The objective of the study is to determine the toxicity effects and toxicokinetic (TK) profile of the test item, aerosolized IL-6R antibody formulation, following 5 inhalation administrations (on Days 1, 4, 7, 10 and 13) to cynomolgus monkeys and to assess the persistence, delayed onset or reversibility of any changes following a 14-day recovery period.

The study will be performed in compliance with GLP regulations.

The test item and vehicle control item will be administered to groups of monkeys by 5 repeated inhalation administrations (on Days 1, 4, 7, 10 and 13) as described in Table 7 below:

TABLE 7
Experimental Design for Inhalation Toxicity Studies in Non-human Primates

| | | Target | | Target | | | | | |
|---|---|---|---|---|---|---|---|---|---|
| | | Delivered Dose | Target Dose of | Aerosol Conc. | | | | | |
| | | Level of IL-6R | IL-6R antibody | of IL-6R | Exposure | Number of Animals | | | |
| Group | Group | antibody | (mg/gram lung | antibody | Duration | Main | | Recovery | |
| No. | Designation | (mg/kg/day) | weight) b | (mg/L) | (min.) | M | F | M | F |
| 1 | Vehicle | 0 | 0 | 0 | 60 | 3 | 3 | 2 | 2 |
| | Control | | | | | | | | |
| 2 | Low Dose | 1.25 | 0.024 | 0.0392 | 60 | 3 | 3 | 2 | 2 |
| 3 | Mid Dose | 6.2 | 0.12 | 0.1954 | 60 | 3 | 3 | 2 | 2 |
| 4 | High Dose | 31 | 0.6 | 0.9800 | 60 | 3 | 3 | 2 | 2 |

ₐ = Target dose levels are calculated based on an estimated body weight of 2.4 kg.
b = Pulmonary deposited dose will provide a target lung dose of 0.024, 0.12 and 0.6 mg/gram lung weight calculated based on 25% pulmonary deposited dose (calculated based on a 13 grams lung weight)
M = males and F = females;
Conc. = concentration;
Min. = minute

The Main animals will be euthanized and subjected to a necropsy examination on Day 14. The Recovery animals will be observed for 14 additional dose-free days, then euthanized and subjected to a necropsy examination on Day 27.

For evaluation of T cell-dependent antibody response (TDAR), all animals will be administered a 10 mg/animal dose of the immunogen, Keyhole Limpet Hemocyanin (KLH), on Day 3 by multiple (4) site subcutaneous injections at a total dose volume of 1 mL/animal (0.25 mL per site).

### Formulation and Inhalation Exposure System

The aerosol will be produced by metering the flow of the IL-6R antibody or vehicle control formulations to 3 clinical nebulizers (Aeroneb Solo). The aerosol produced will be discharged through a 40 mm diameter tube into a flow-past inhalation exposure system. The airflow rate through the exposure system will be monitored and recorded manually during the aerosol generation. Airflow to the exposure system will be controlled by the absolute volume of air supplying the aerosol generators using variable area flow meters. Control of the aerosol exhaust flow from the animal exposure system will be achieved using an exhaust valve. The system (7 open ports) will provide a minimum aerosol of 3 L/minute for Groups 2 and 3, and 4 L/minute for Groups 1 and 4, to each animal exposure position; and the inlet and outlet airflows will be balanced to ensure that there is no dilution of the generated aerosol by air drawn from the environment. Any minor variations in flow will be buffered by a balloon reservoir. An equal delivery of aerosol to each exposure position will be achieved by employing a distribution network that is identical for each individual exposure position attached to the system.

### Measurements and Analysis

A series of 6 blood samples (approximately 0.5 mL each) will be collected from each monkey on Day 1 pre-dose and at 2, 6, 24, 48 and 72-hours after dosing. A series of 3 blood samples (approximately 0.5 mL each) will be collected from each monkey on Day 4 pre-dose and at 3 and 7-hours after the end of dosing and on Day 13 pre-dose and at 4 and 8 hours after the end of dosing. One blood sample will be collected at the end of the recovery period. For this purpose, each monkey will be bled by venipuncture and the samples will be collected into tubes containing clotting activator.

Following collection, blood samples for serum will be allowed to stand at room temperature for approximately 30 minutes to clot, the samples will be centrifuged (2500 rpm for 10 minutes at approximately 4° C.), and the resulting serum will be recovered, divided into two aliquots (sets 1 and 2) and stored frozen (≤−60° C.) in appropriately labelled vials or tubes.

### Broncho-Alveolar Lavage (BAL) Fluid and Cell Collection, Processing and Bioanalysis

BAL will be performed from all animals at termination. Each animal will be sacrificed and the lungs will then be removed from the animal. BAL fluid collection will be completed within 1 hour after BAL collection. The BAL samples will be centrifuged at approximately 800 g for 10 minutes at approximately 4° C. to precipitate the cells. After centrifugation, the lavage fluid will be transferred to another suitable centrifugation tube (to avoid disturbing the cell pellet) and centrifuged again at 2000-2500 g (4° C.) for 10 minutes to precipitate cell debris. Aliquots of each supernatant fluid will be divided into 2 separate tubes: one sample will be analyzed for test item IL-6R antibody and another sample will be analyzed for antibodies against the IL-6R antibody (ADA). The sample for IL-6R antibody analysis will be analyzed using a validated ELISA and the ADA sample will be stored frozen (≤600C) for future possible analysis.

The toxicokinetic parameters will be calculated. The following configuration will be used for the analysis:
Sampling Method: Rich
AUC Calculation Method: Linear Trapezoidal with Linear Interpolation.
Lambda Z (Xz) Method: Best fit for Xz, Log regression
Weighting (Xz calculation): Uniform
Toxicokinetic parameters (including abbreviation and description for each parameter) are described in Table 8

TABLE 8
Summary of Toxicokinetic and Pharmacokinetic Parameters

| Parameters | Abbreviation |
|---|---|
| Area under the plasma drug concentration-time curve from | AUC0-Tlast |
| the time of dosing to the last quantifiable concentration | |
| Area under the plasma drug concentration-time curve from | AUC_{INF} |
| the time of dosing extrapolated to infinity | |
| Terminal elimination half-life | T_{1/2} |
| The maximum plasma concentration | Cₘₐₓ |
| Time to maximum plasma concentration | Tₘₐₓ |

Results from this study will determine the toxicity and pharmacokinetic profile of the aerosolized IL-6R antibody administered by a clinical nebulizer to non-human primates.

### Example 4: Dose Delivered by Aerosolization in Non-Human Primates

Animals were dosed 60 for minutes with aerosolized IL-6R antibody at the low dose, medium dose, and high dose as described in Example 3. The lung tissue was analyzed for presence of the IL-6R antibody and aerosol particle size was analyzed.

Following 13 days of dosing, no serious clinical signs were observed. Two animals showed clinical signs in the form of respiratory distress and cyanosis, and each recovered.

The achieved lung doses were near target dose levels and particle size analysis show the aerosolized formulation is within target size range (Table 9).

TABLE 9
Summary of Particle Size and Amount of IL-6R Antibody Administered
to the Lung by Aersolization in Non-human Primates

| | | Target | Achieved | | |
|---|---|---|---|---|---|
| | | lung | lung | | |
| | | (mg/gram | (mg/gram | Particle Size Data | |
| Day | Group | of lung) | of lung) | MMAD (μm) | sg |
| 1 | 0 (Control) | 0 | 0 | — | — |
| | 2 (low dose) | 0.024 | 0.030 | 2.1 | 2.50 |
| | 3 (mid dose) | 0.12 | 0.13 | 2.3 | 2.29 |
| | 4 (High dose) | 0.60 | 0.56 | 2.5 | 2.32 |
| 4 | 0 (Control) | 0 | 0 | — | — |
| | 2 (low dose) | 0.024 | 0.030 | 3.7 | 3.44 |
| | 3 (mid dose) | 0.12 | 0.13 | 2.8 | 2.5 |
| | 4 (High dose) | 0.60 | 0.56 | 3.3 | 2.49 |
| 7 | 0 (Control) | 0 | 0 | — | — |
| | 2 (low dose) | 0.024 | 0.027 | 2.3 | 2.69 |
| | 3 (mid dose) | 0.12 | 0.117 | 2.5 | 2.39 |
| | 4 (High dose) | 0.60 | 0.50 | 2.7 | 2.40 |
| 10 | 0 (Control) | 0 | 0 | — | — |
| | 2 (low dose) | 0.024 | 0.023 | 1.8 | 2.05 |
| | 3 (mid dose) | 0.12 | 0.123 | 1.6 | 2.04 |
| | 4 (High dose) | 0.60 | 0.50 | 2.7 | 2.19 |
| 13 | 0 (Control) | 0 | 0 | — | — |
| | 2 (low dose) | 0.024 | 0.026 | 2.1 | 2.66 |
| | 3 (mid dose) | 0.12 | 0.11 | 2.3 | 2.6 |
| | 4 (High dose) | 0.60 | 0.52 | 2.2 | 2.99 |

MMAD = Mass Median Aerodynamic Diameter

These results indicate that IL-6R antibody can be delivered by aerosolization using nebulizer, to the lung of non-human primates with reliable and predictable dose accuracy using Formulation F5 (20 mg/mL anti-IL-6 antibody, 25 mM His, 125 mM NaCl, 0.05% PS80, pH 6.0).

1. A method of treating, preventing, or alleviating a symptom of a coronavirus infection in a subject in need thereof comprising administering to the subject a composition comprising an IL-6R antibody,
wherein the IL-6R antibody comprises a VH CDR1 region comprising the amino acid sequence of SEQ ID NO: 15, a VH CDR2 region comprising the amino acid sequence of SEQ ID NO: 37, a VH CDR3 region comprising the amino acid sequence of SEQ ID NO: 35, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26; and
wherein the composition is administered by inhalation.

2.-4. (canceled)

5. The method of claim 1, wherein the IL-6R antibody is a monoclonal antibody.

6. The method of claim 5, wherein the antibody is fully human.

7. (canceled)

8. The method of claim 1, wherein the coronavirus is COVID-19, SARS or MERS.

9. The method of claim 1, where the symptom of a coronavirus infection is cytokine release syndrome, acute respiratory-distress syndrome (ARDS), fever, cough, or shortness of breath.

10. The method of claim 1, wherein the subject has or is suspected of having a coronavirus infection.

11. The method of claim 1, wherein the subject has been or thought to have been exposed to a coronavirus and has not yet developed symptoms of a coronavirus infection.

12. (canceled)

13. The method of claim 1, wherein the inhalation administration is by an inhaler or a nebulizer.

14. The method of claim 13, wherein the inhaler or the nebulizer comprises a composition comprising the IL-6R antibody, histidine, sodium chloride, and polysorbate 80.

15. The method of claim 14, wherein the histidine is about 5 mM, about 10 mM, about 15 mM, about 20 mM, about 25 mM, about 30 mM, about 35 mM, or about 40 mM.

16. (canceled)

17. The method of claim 14, wherein the sodium chloride is about 50 mM, about 75 mM, about 100 mM, about 125 mM, about 150 mM, about 175 mM or about 200 mM.

18. (canceled)

19. The method of claim 14, wherein the polysorbate 80 is about 0.01%, about 0.02%, about 0.03%, about 0.04%, about 0.05%, about 0.06%, about 0.07%, about 0.08%, about 0.09%, or about 0.1%.

20.-21. (canceled)

22. The method of claim 14, wherein the composition has a pH of about 5.0, about 6.0, or about 7.0.

23. (canceled)

24. The method of claim 14, wherein the IL-6R antibody is about 5 mg/mL, about 10 mg/mL, about 15 mg/mL, about 20 mg/mL, about 25 mg/mL, about 30 mg/mL, or about 35 mg/mL.

25.-40. (canceled)

41. A composition comprising an IL-6R antibody, histidine, sodium chloride, and polysorbate 80.

42.-57. (canceled)

53. The composition of claim 41, wherein the histidine is 25 mM, the sodium chloride is 125 mM, the polysorbate 80 is 0.02%, the pH is 6.0, and the IL6R antibody is 20 mg/mL.

58. A method of treating, or alleviating a symptom of a pulmonary inflammatory disease in a subject in need thereof comprising administering to the subject a composition comprising an IL-6R antibody.

59. (canceled)

60. The method of claim 58, wherein the pulmonary inflammatory disease is selected from acute respiratory distress syndrome (ARDS) or systemic pulmonary sclerosis.
